(19) United States
(12) Patent Application Publication
(10) Pub. No.: US 2007/0191357 A1
(43) Pub. Date: Aug. 16, 2007

(54) METHODS OF USING POTASSIUM CHANNEL INHIBITING COMPOUNDS

(76) Inventors: Jochen Antel, Bad Munder (DE); Peter-Colin Gregory, Hannover (DE); Michael Firnges, Barsinghausen (DE); Dania Reiche, Adelheidsdorf (DE)

Correspondence Address: MAYER, BROWN, ROWE & MAW LLP, 71 S. WACKER, CHICAGO, IL, 60606, US

(21) Appl. No.: 11/465,258

(22) Filed: Aug. 17, 2006

Related U.S. Application Data
(60) Provisional application No. 60/708,940, filed on Aug. 17, 2005.

(51) Int. Cl. A61K 31/425

(52) U.S. Cl. 514/230.2; 514/222.8; 514/341; 514/372

(57) ABSTRACT

Described herein are methods of treating, preventing or inhibiting a variety of medical conditions by administering to subjects in need thereof an effective amount of at least one potassium K_{v1.3} channel inhibiting compound which can optionally also have either or both CBₓ modulating properties and/or potassium K₍ₐₜₚ₎ channel opening properties. Also described is the use of potassium K_{v1.3} channel inhibiting compounds in a pharmaceutical composition for the prophylaxis, treatment, delayed progression, delayed onset and/or inhibition of a variety of medical conditions.

### CROSS REFERENCE TO RELATED APPLICATIONS

This application claims the benefit of U.S. Provisional Application No. 60/708,940 filed Aug. 17, 2005 which is hereby incorporated by reference.

### FIELD OF THE INVENTION

Described herein are methods of treating, preventing or inhibiting various medical conditions, such as type I and type II diabetes, by administering an effective amount of at least one potassium K_{v1.3} channel inhibitor to subjects in need thereof. Optionally, the potassium K_{v1.3} channel inhibitor can also have either or both CBₓ modulator and/or a potassium K₍ₐₜₚ₎ channel opening properties.

### BACKGROUND OF THE INVENTION

Insulin is a critical modulator of glucose and lipid homeostasis and cellular proliferation. It is secreted into the bloodstream by the pancreatic β-cells in response to a rise in serum glucose and amino acids, such as occurs following meal ingestion, but is also secreted as part of the pre-absorptive, cephalic phase of meal ingestion. This insulin binds to a specific insulin receptor (IR) at the plasma membrane of cells of insulin-responsive tissues, such as skeletal muscle, fat and liver. Brain cells expressing IR are believed to play a role in glucose homeostasis and appetite regulation. Binding of insulin to IR initiates a cascade of events that result in translocation of the glucose transporter GLUT4 to the plasma membrane e.g. of skeletal (and cardiac) muscle and adipocytes, or of GLUT2 to the plasma membrane of hepatocytes and this allows glucose uptake into the cell and its metabolism.

Type II diabetes (non-insulin-dependent diabetes mellitus or “NIDDM”) patients display a gradually increasing degree of insulin resistance. Early in the disease, insulin secretion is typically increased in an effort to maintain normal glucose metabolism but as the disease progresses, insulin secretion falls because of the chronic overstimulation of the pancreatic islets. At this late stage, NIDDM patients compare to type I diabetes (insulin-dependent diabetes mellitus or “IDDM”) patients, in that they do not produce enough insulin to maintain normal glucose metabolism. Present therapy for NIDDM, in addition to diet and exercise, comprises monotherapy or combination therapy with insulin-releasing agents (e.g. sulphonylureas) or injectable insulin, insulin-sensitizing agents (such as metformin, or the TDZ's), alpha-glucosidase inhibitors (e.g., acarbose) or lipase inhibitors (e.g., Xenical®). Therapy of type I diabetes (IDDM) requires injectable insulin, diet and exercise.

The underlying causes of insulin resistance are the subject of intense research, but strongly implicated is an increase in plasma free fatty acid levels, which is believed to play a key role in development of insulin resistance, Ferrannini et al, “Effect of fatty acids on glucose production and utilization in man”, J. Clin. Invest., 72: 1737-1747 (1983), probably by reducing glucose transport into the cells. Dresner et al, “Effects of free fatty acids on glucose transport and IRS-1—associated phosphatidylinositol 3-kinase activity,” J. Clin. Invest., 103: 253-259 (1999). In addition, e.g., in obesity, the release of inflammatory cytokines such as tumor necrosis factor-alpha (TNF-α) and Interleukin-6 (Il-6) from adipose tissue appear to be involved in development of insulin resistance, perhaps via activation of c-jun N-terminal kinase (JNK). Hirosumi et al, “A central role for JNK in obesity and insulin resistance,” Nature, 420: 333-336 (2002).

The incidence of NIDDM continues to increase alarmingly and there is a clear need for new methods of treating obese- and non-obese type I and type II diabetes. It has now surprisingly been found that the administration of potassium K_{v1.3} channel inhibitors significantly improve the medical condition of obese- and non-obese diabetes type I and type II patients.

The voltage gated potassium K_{v1.3} channel, which belongs to the Shaker family of Kᵥ channels that regulate cell membrane potential, is expressed in many tissues, including lymphocytes, kidney, adipocytes and skeletal muscle. It has six transmembrane domains, S1-S6, and a pore region. It contains consensus sequences for a protein kinase C (PKC) site between S4 and S5, which is believed to play an important role in channel function, a tyrosine kinase phosphorylation site at the amino terminus and an N-glycosylation site between S1 and S2.

PKC increases and tyrosine kinase (TK) inhibits potassium K_{v1.3} channel activity. Chung & Schlichter, “Native K_{v1.3} channels are up-regulated by protein kinase C,” J. Membr. Biol., 156: 73-85 (1997); Fadool et al, “Brain insulin receptor causes activity-dependent current suppression in the olfactory bulb through multiple phosphorylation of K_{v1.3},” J. Neurophysiol., 83: 2332-2348 (2000). Furthermore, channel activity is upregulated by serum-glucocorticoid activated kinase, and at least in olfactory bulb neurons, the brain region with the highest insulin binding, its activity is downregulated by insulin via activation of receptor TK. Fadool et al, 2000.

It has been found that potassium K_{v1.3} channel inhibitors increase metabolic rate. Xu et al, “The voltage-gated potassium channel K_{v1.3} regulates energy homeostasis and body weight,” Human Molecular Genetics, 12: 551-559 (2003). Furthermore, inhibition of potassium K_{v1.3} increases peripheral insulin sensitivity. Xu et al, “The voltage-gated potassium channel K_{v1.3} regulates peripheral insulin sensitivity,” Proc. Nat. Acad. Sci, 101: 3112-3117 (2004). This effect is primarily due to an increase in glucose uptake in fat and skeletal muscle, which in turn can be mainly attributed to an increase in translocation of the GLUT4 glucose transporter (the major transporter mediating glucose uptake in insulin-sensitive tissues) from intracellular stores to the plasma membrane of skeletal muscle and adipose cells. In addition, potassium K_{v1.3} channel inhibition reduces production of Il-6 and TNF-α by adipocytes and decreases JNK activity, which further helps to improve insulin sensitivity. Xu et al, 2004. Therefore, potassium K_{v1.3} channel inhibition is suited for both treatment and prophylaxis of NIDDM.

It is now further been found that development of IDDM appears to involve auto-immune destruction of pancreatic beta cells. Lemmark, “Type 1 Diabetes—does suppressing T cells increase insulin?” N. Engl. J. Med., 352 (25): 2642-2644 (2005). Potassium K_{v1.3} channel blockade selectively suppresses the activation and proliferation of effector memory T-cells while sparing activation of naive or T central memory cells, Venne-kamp et al, “K_{v1.3}-blocking 5-phenylalkoxypsoralens: A new class of immunomodulators.” Mol. Pharmacol., 65: 1364-1374 (2004); Damjanovich Gaspar & Panyi, “An alternative to conventional immunosuppression: small-molecule inhibitors of K_{v1.3} channels,” Mol. Interv., 4 (5) 250-254 (2004), and thus offers great promise for therapy of IDDM patients with residual insulin secretion, by halting islet cell destruction and progression of the disease and reducing the need for insulin injection by prolonging the period of insulin secretion. Moreover, it may also cause further benefit in IDDM by allowing control of blood glucose with lower doses of insulin due to the improved insulin sensitivity. The selective immunosuppressive actions of potassium K_{v1.3} blockade also offer promise for therapy of other auto-immune diseases such as multiple sclerosis, chronic graft rejection and graft-versus-host disease.

### SUMMARY

Accordingly, one embodiment disclosed herein includes administering to subjects in need thereof an effective amount of a at least one potassium K_{v1.3} channel inhibitor which optionally has either or both CBₓ modulating properties and/or potassium K₍ₐₜₚ₎ channel opening properties.

Another embodiment disclosed herein includes the use of an effective amount of at least one potassium K_{v1.3} channel inhibitor which optionally has either or both CBₓ modulating properties and/or potassium K₍ₐₜₚ₎ channel opening properties for the manufacture of a pharmaceutical composition for the prophylaxis, treatment, delayed progression, delayed onset and/or inhibition of a variety of medical conditions.

Other objects, features and advantages will be set forth in the detailed description of the embodiments that follows, and in part will be apparent from the description or may be learned by practice of the claimed invention. These objects and advantages will be realized and attained by the processes and compositions particularly pointed out in the written description and claims hereof.

### BRIEF DESCRIPTION OF THE DRAWINGS

FIG. 1 shows the stimulation protocol for investigation of the test compound.

FIG. 2 shows the test item application protocol

FIG. 3 shows the K_{v1.3} mediated potassium current.

FIG. 4A shows 80 superimposed original potassium K_{v1.3}-current traces recorded in the presence and absence of 10 μM of example compound 1.

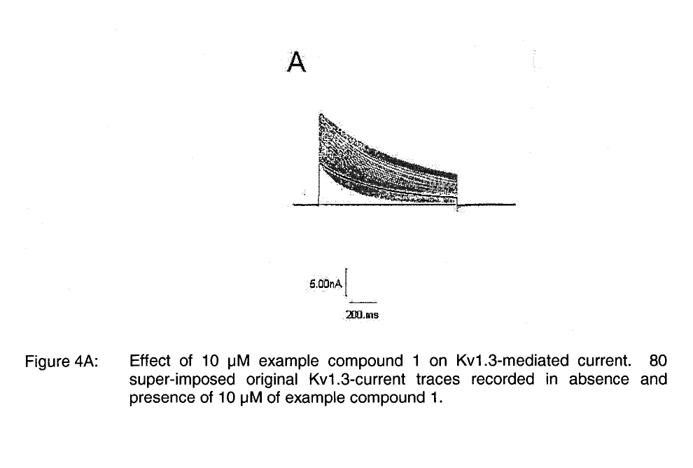

FIG. 4B shows current amplitude plotted against time. Onset (indicated by the long dashed line) and offset (indicated by the short dashed line) of test compound application. Extrapolated time course of current amplitude under vehicle conditions was calculated by a biexponential fit of equation Y=a*exp(−cx)+b*exp(−dx) and is depicted as the solid line.

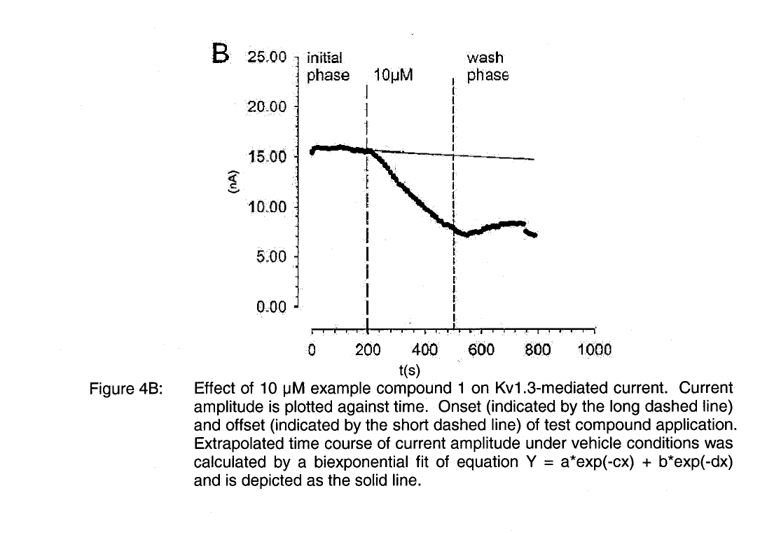

FIG. 5 shows the concentration-dependence effect of example compound 1 on the K_{v1.3}-mediated potassium current.

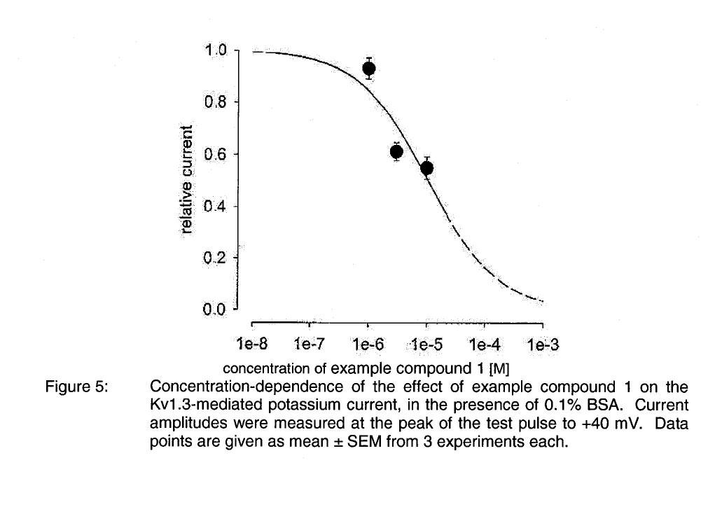

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. The headings used through-out this disclosure are provided for convenience only and are not to be construed to limit the invention in any way. Embodiments illustrated under any heading may be combined with embodiments illustrated under any other heading.

Compounds having CBₓ modulating properties are preferably selected from the group consisting of: CB₁ antagonists, CB₁ agonists and CB₂ agonists.

Compounds which inhibit the potassium K_{v1.3} channel greater than about 40%, preferably greater than about 60%, more preferably greater than about 80%, even more preferably greater than about 90% and most preferably greater than about 95% or above, are suitable potassium K_{v1.3} channel inhibiting compounds.

As used herein, the term “various medical conditions” shall include but not be limited to obesity, diabetes mellitus, metabolic syndrome, syndrome X, insulinoma, familial hyperinsulemic hypoglycemia, male pattern baldness, detrusor hyperreactivity, asthma, glucose metabolism—in particular, insulin resistance, hyperglycaemea and/or glucose intolerance—neuroprotection, epilepsy, analgesia, cardioprotection, angina, cardioplegia, arrhythmia, coronary spasm, peripheral vascular disease, cerebral vasospasm, appetite regulation, neurodegeneration, pain—including neuropathic pain and chronic pain—and impotence.

Described herein are methods of treating, preventing or inhibiting various medical conditions.

In one embodiment, methods of treating, preventing or inhibiting various medical conditions by administering an effective amount of at least one potassium K_{v1.3} channel inhibitor to subjects in need thereof are described. It has been found that patients, subject to treatment with an effective amount of at least one potassium K_{v1.3} channel inhibitors show an improved glycemic control and insulin management. In this embodiment, an effective amount of at least one potassium K_{v1.3} channel inhibitor is employed.

In another embodiment, methods of treating, preventing or inhibiting various medical conditions by administering an effective amount of at least one compound having potassium K_{v1.3} channel inhibiting properties and also optionally having either or both CBₓ modulating properties and/or potassium K₍ₐₜₚ₎ channel opening properties to subjects in need thereof are described. It has been found that patients, subject to treatment with such compound(s) show an improved glycaemic control and insulin management. In this embodiment, an effective amount of such compound(s) is employed.

In another embodiment, methods of treating, preventing or inhibiting various medical conditions by administering an effective amount of at least one compound having both potassium K_{v1.3} channel inhibiting properties and CBₓ modulating properties to subjects in need thereof are described. It has been found that patients, subject to treatment with an effective amount of such compound(s) show an improved glycaemic control and insulin management. In this embodiment, an effective amount of such compound(s) is employed.

In another embodiment, methods of treating, preventing or inhibiting various medical conditions by administering an effective amount of at least one compound having both potassium K_{v1.3} channel inhibiting properties and potassium K₍ₐₜₚ₎ channel opening properties to subjects in need thereof are described. It has been found that patients, subject to treatment with such compound(s) having both potassium K_{v1.3} channel inhibiting properties and potassium K₍ₐₜₚ₎ channel opening properties show an improved glycaemic control and insulin management. In this embodiment, an effective amount of at least one compound having both potassium K_{v1.3} channel inhibiting properties and potassium K₍ₐₜₚ₎ channel opening properties is employed.

In another embodiment, methods of treating, preventing or inhibiting various medical conditions by administering an effective amount of at least one compound having potassium K_{v1.3} channel inhibiting properties, CBₓ modulating properties and potassium K₍ₐₜₚ₎ channel opening properties, to subjects in need thereof are described. It has been found that patients, subject to treatment with an effective amount of at least one of such compound(s) show an improved glycaemic control and insulin management. In this embodiment, an effective amount of at least one such compound having potassium K_{v1.3} channel inhibiting properties, CBₓ modulating properties and potassium K₍ₐₜₚ₎ channel opening properties is employed.

In a specific embodiment, obese- and non-obese type I and type II diabetes and related conditions are treated, prevented or inhibited. In an even more specific embodiment, the related condition is glucose metabolism, such as, e.g., insulin resistance, hyperglycemia and/or glucose intolerance.

In the methods and uses described herein, any potassium K_{v1.3} channel inhibitor optionally having in addition either or both CBₓ modulating properties and/or potassium K₍ₐₜₚ₎ channel opening properties, can be utilized for the purposes described herein. The following compounds are preferred:
a.) 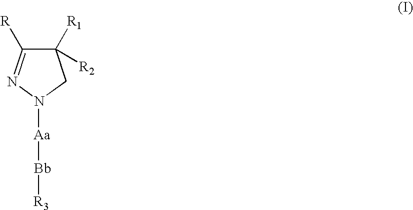
wherein:
   R and R₁ are independently selected from the group consisting of: napthyl, phenyl, thienyl and pyridyl wherein phenyl, thienyl and pyridyl may be substituted with 1, 2 or 3 substitutents Y;
   R₂ is selected from the group consisting of: hydrogen, hydroxy, C₁₋₃-alkoxy, acetyloxy and propionyloxy;
   R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
   Aa is selected from the group consisting of: substitutents of formulae (i), (ii), (iii), (iv), (v) and (vi) 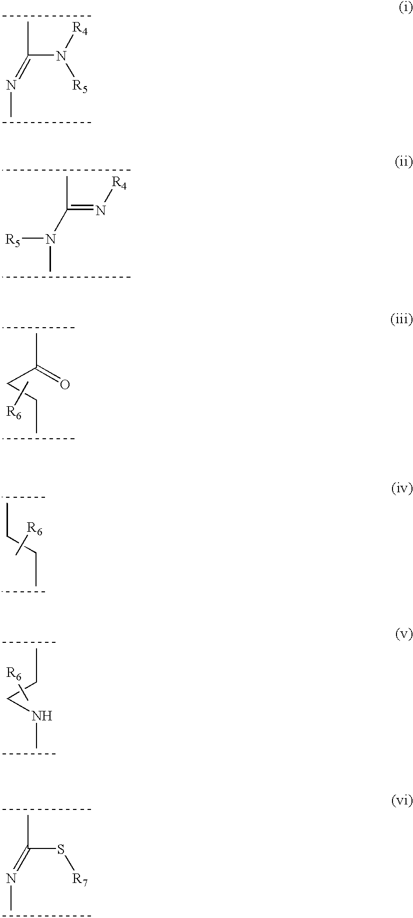
   Bb is selected elected from the group consisting of: sulfonyl and carbonyl;
   each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoro-methylsulfonyl, cyano, carbamoyl, sulfamoyl and acetyl;
   R₄ is selected from the group consisting of: hydrogen, C₁₋₈ branched or unbranched alkyl, C₃₋₈ cycloalkyl; or R₄ is selected from the group consisting of: acetamido, dimethylamino, 2,2,2-trifluoroethyl, phenyl and pyridyl with the proviso that R₅ is hydrogen,
   wherein such C₁₋₈ branched or unbranched alkyl and/or C₃₋₈ cycloalkyl alkyl group may be substituted with a hydroxyl group;
   R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
   wherein such C₁₋₈ branched or unbranched alkyl and/or C₃₋₈ cycloalkyl group may be substituted with hydroxyl and/or fluoro,
   wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
   wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
   wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₃ alkyl, hydroxy, amino, monoalkylamino, dialkylamino or fluoro,
   wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
   wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
   wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
   wherein such phenyl, benzyl, pyridyl, thienyl, pyridylmethyl or phenethyl group may be substituted with 1, 2 or 3 of the substitutents Y,
   wherein such monocyclic or bicyclic heterocyclic moiety having 4 to 10 ring atoms may contain one or more heteroatoms selected from the group consisting of: O, N and S,
   wherein such monocyclic or bicyclic heterocyclic moiety having 4 to 10 ring atoms may contain a keto or —SO₂— group,
   wherein such monocyclic or bicyclic heterocyclic moiety having 4 to 10 ring atoms may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
   R₆ is selected from the group consisting of: hydrogen and C₁₋₃ unbranched alkyl;
   R₇ is C₁₋₃ unbranched alkyl;
   R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
   wherein such saturated or unsaturated heterocyclic moiety having 4 to 8 ring atoms may contain an additional heteroatom selected from the group consisting of: N, O and S or may contain a group selected from the group consisting of: a keto or —SO₂— group,
   wherein such saturated or unsaturated heterocyclic moiety having 4 to 8 ring atoms may be substituted with C₁₋₄ alkyl;
   R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁—together with the nitrogen atom to which they are bonded—form a monocyclic, bicyclic or tricyclic alkyl or alkenyl group,
   wherein such branched or unbranched C₁₋₈ alkyl and/or branched or unbranched C₁₋₈ alkenyl groups may contain one or more heteroatoms selected from the group consisting of: O, N, and S,
   wherein such branched or unbranched C₁₋₈ alkyl and/or branched or unbranched C₁₋₈ alkenyl groups may contain a group selected from the group consisting of: keto and —SO₂— group and wherein such keto and —SO₂— group may be substituted with a hydroxy or amino group,
   wherein such C₃₋₈ cycloalkyl and/or C₃₋₈ cycloalkenyl group may contain one or more ring heteroatoms selected from the group consisting of: O, N, and S,
   wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
   wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
   wherein such monocyclic, bicyclic or tricyclic alkyl or alkenyl group may contain ring heteroatoms selected from the group consisting of: O, N and S,
   wherein such monocyclic, bicyclic or tricyclic alkyl or alkenyl group may contain a group selected from the group consisting of: keto and SO₂,
   wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
   wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 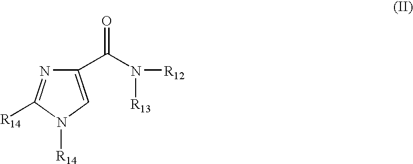
wherein
   R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
   R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 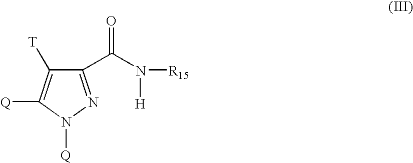
wherein
   Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
   T is selected from the group consisting of: hydrogen, C₁₋₃ alkyl and C₃₋₆ cycloalkyl which may contain from 1 to 3 heteroatoms selected from the group consisting of: N, O and S;
   R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and a prodrug thereof, a tautomer thereof or a pharmaceutically acceptable salt thereof;
e.) and mixtures thereof.

More preferred are 4,5-dihydro-1H-pyrazole derivatives of the formula (I), prodrugs, tautomers or pharmaceutically acceptable salts thereof; 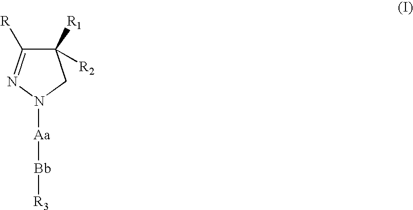
wherein the 4-position of the 4,5 dihydro pyrazole ring is in the S-configuration.

Compounds inhibiting the potassium K_{v1.3} channel by greater than about 40% include the following: 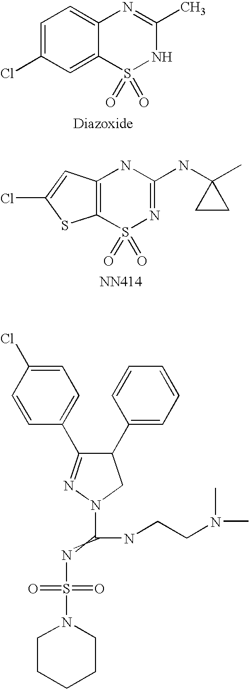
Compounds inhibiting the potassium K_{v1.3} channel by greater than about 60% include the following: 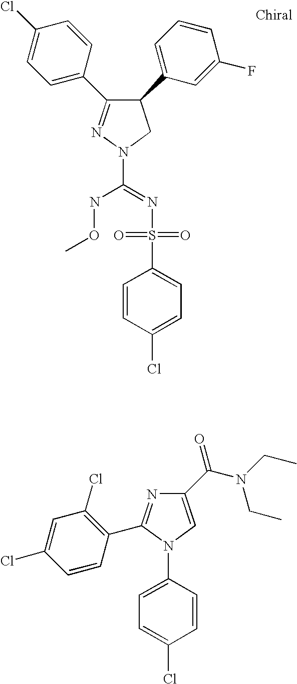 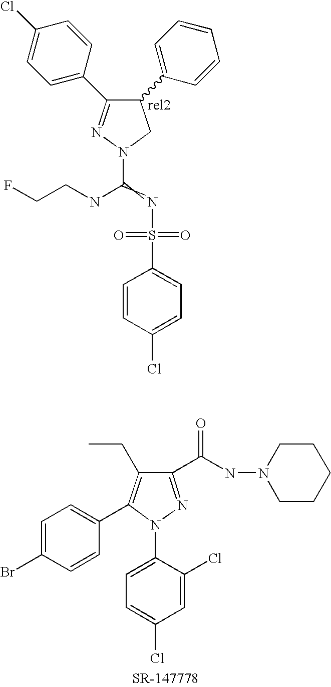
Compounds inhibiting the potassium K_{v1.3} channel by greater than about 80% include the following: 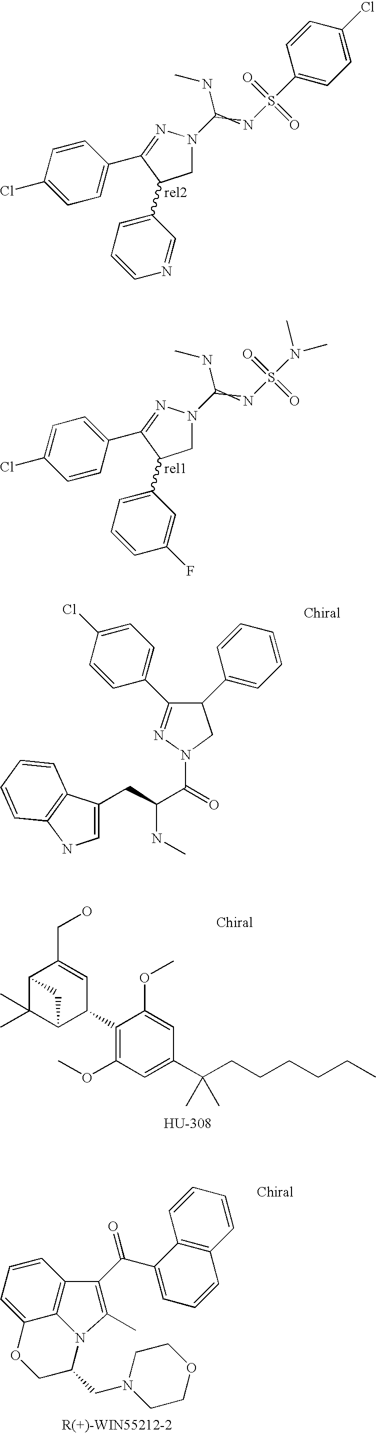 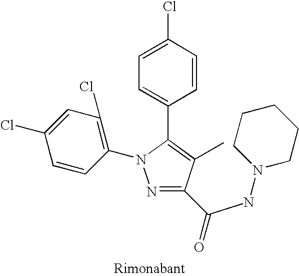
Compounds inhibiting the potassium K_{v1.3} channel by greater than about 90% include the following: 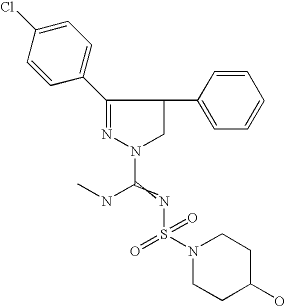
Compounds inhibiting the potassium K_{v1.3} channel by greater than about 95% include the following:

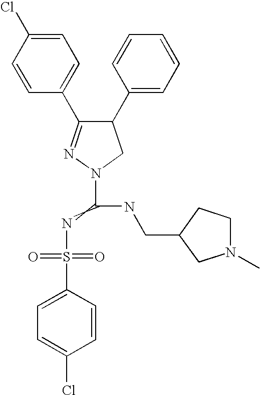

All the above compounds are effective potassium K_{v1.3} channel inhibitors and optionally have in addition one or both CBₓ modulating properties or potassium K₍ₐₜₚ₎ channel opening properties. CBₓ modulating properties are preferably selected from the group consisting of: CB₁ antagonists, CBₓ agonists and CB₂ agonists.

Combination with CB₁ Antagonist:

In a another embodiment, methods of treating obese- and non-obese type I and type II diabetes and related conditions by administering an effective amount of at least one potassium K_{v1.3} channel inhibitor in combination with an effective amount of at least one CB₁ antagonist to subjects in need thereof are described. It has been found that obese diabetes type I and type II patients, subject to treatment with an effective amount of at least one potassium K_{v1.3} channel inhibitor in combination with an effective amount of at least one CB₁ antagonist, show a significantly improved glycaemic control and insulin management.

In still another embodiment, methods of treating obese- and non-obese type I and type II diabetes and related conditions by administering an effective amount of at least one compound having both potassium K_{v1.3} channel inhibiting properties and CB₁ antagonistic properties in combination with an effective amount of at least one CB₁ antagonist to subjects in need thereof are described. It has been found that obese diabetes type I and type II patients, subject to treatment with an effective amount of at least one compound having both potassium K_{v1.3} channel inhibiting properties and CB₁ antagonistic properties, in combination with an effective amount of at least one CB₁ antagonist, show a significantly improved glycaemic control and insulin management.

Obesity is a major cause of NIDDM and the combination of a CB₁ antagonist, which causes weight loss principally by reduced food intake, with a potassium K_{v1.3} channel inhibitor, which increases metabolic rate (Xu et al 2003) as well as directly improving insulin sensitivity is particularly suited for the prophylaxis and therapy of NIDDM.

Any CB₁ antagonist known in the art can be utilized for the purpose described herein. Suitable CB₁ antagonists are, e.g., those which are useful to treat appetite disorders and/or obesity, e.g. SR 147778. A review is given in J. H. M. Lange and C. G. Kruse, Current Opinion in Drug Discovery & Development 7(4) (2004) 498-506. Further examples of such compounds are described in documents U.S. Pat. No. 5,624,941; U.S. Pat. No. 6,344,474; U.S. Pat. No. 6,509,367; WO 01/032663; WO 01/070700; WO 03/007887; WO 03/015700; WO 03/026647; WO 03/026648; WO 03/027076; WO 03/040107; WO 03/051850; WO 03/051851; WO 03/063781; WO 03/077847; WO 03/078413; WO 03/082190; WO 03/082191; WO 03/082256; WO 03/082833; WO 03/084930; WO 03/084943; WO 03/086288; WO 03/087037; WO 03/088968; WO 04/012671; WO 04/013120; WO 04/026301; WO 04/052864; WO 04/060888; WO 04/060870; WO 04/058727 and WO 04/058255, the contents of which are herewith incorporated by reference.

Combination with Potassium K₍ₐₜₚ₎ Channel Opener:

In another embodiment, methods of treating obese- and non-obese type I and type II diabetes and related conditions by administering an effective amount of at least one potassium K_{v1.3} channel inhibitor in combination with an effective amount of at least one potassium K₍ₐₜₚ₎ channel opener to subjects in need thereof are described. It has been found that obese- and non-obese diabetes type I patients, subject to treatment with an effective amount of at least one potassium K_{v1.3} channel inhibitor in combination with an effective amount of at least one potassium K₍ₐₜₚ₎ channel opener, show a significantly improved glycaemic control and insulin management.

Still another embodiment, methods of treating obese- and non-obese type I and type II diabetes and related conditions by administering an effective amount of at least one compound having both potassium K_{v1.3} channel inhibiting properties and CB₁ antagonistic properties in combination with an effective amount of at least one potassium K₍ₐₜₚ₎ channel opener to subjects in need thereof are described. It has been found that obese- and non-obese diabetes type I patients, subject to treatment with an effective amount of at least one compound having both potassium K_{v1.3} channel inhibiting properties and CB₁ antagonistic properties in combination with an effective amount of at least one potassium K₍ₐₜₚ₎ channel opener show a significantly improved glycaemic control and insulin management.

Reduction of insulin secretion by a SUR1 potassium K₍ₐₜₚ₎ channel opener, which may need to be combined at least initially with insulin therapy, protects against over-stimulation of the pancreatic islets as in early stage NIDDM the β-cells attempt to overcome the developing insulin resistance by increased production of insulin. The reduced metabolic strain on the islet cells leads to improved function of the β-cell. Guldstrand et al, “Improved β-cell function after short term treatment with diazoxide in obese patients with Type 2 diabetes,” Diabetes Metab., 28: 448-456 (2002). Chronic therapy with a SUR1 potassium K₍ₐₜₚ₎ channel opener also leads to improved insulin sensitivity, perhaps via reduction of hepatic gluconeogenesis. Pocal et al, “Hypothalamic K₍ₐₜₚ₎ channels control hepatic glucose production,” Nature, 434: 1026-1031 (2005). The combination of SUR1 potassium K₍ₐₜₚ₎ channel opener and potassium K_{v1.3} channel inhibitor will therefore be especially beneficial for treatment and prophylaxis of NIDDM. It may also benefit IDDM patients with residual insulin secretion.

Potassium K₍ₐₜₚ₎ channel openers and their potential use in the inhibition of insulin secretion and/or the treatment of metabolic disorders are known from various references, such as U.S. Pat. No. 6,492,130; WO 02/00223; WO 02/00665 or from Carr et al., Diabetes, 52:2513-2518 (2003) or Hansen et al., Current Medicinal Chemistry, 11:1595-1615 (2004).

The beneficial role of the specific potassium K₍ₐₜₚ₎ channel opener diazoxide in the treatment of metabolic syndrome is known from various references, such as U.S. Pat. No. 5,284,845 or U.S. Pat. No. 6,197,765 or from R. Alemzadeh et al., Endocrinology 133 (2) (1993) 705-712 or Alemzadeh et al., Journal of Clinical Endocrinology and Metabolism, 83(6):1911-1915 (1998).

Any potassium K₍ₐₜₚ₎ channel opener known to one of skill in the art can be utilized for the purpose described herein. Suitable potassium K₍ₐₜₚ₎ channel openers are preferably compounds which have effects as openers at the Kir6.2/SUR1 potassium K₍ₐₜₚ₎ channel, at the Kir6.2/SUR2B potassium K₍ₐₜₚ₎ channel and/or the Kir6.1/SUR2B potassium K₍ₐₜₚ₎ channel. Effective compounds are those which exhibit an IC₅₀ value [μmol] of less than 50 in a test for the affinity of the compounds in binding to the sulfonylurea (=SUR) and potassium channel opener site (═KCO) of rat and/or human isoforms of SUR1 and/or SUR2B—e.g. the test model provided below. Compounds with an effect as openers at the Kir6.2/SUR1 potassium K₍ₐₜₚ₎ channel, in particular as selective openers at the Kir6.2/SUR1 potassium K₍ₐₜₚ₎ channel are preferred. A compound with an effect as opener at the Kir6.2/SUR1 potassium K₍ₐₜₚ₎ channel is understood to be selective if its IC₅₀ value at the Kir6.2/SUR1 potassium K₍ₐₜₚ₎ channel, as measured in the aforementioned binding test, is at most half, more preferred only a quarter, of the IC₅₀ value of that same compound at the Kir6.2/SUR2B potassium K₍ₐₜₚ₎ channel and/or the Kir6.1/SUR2B potassium K₍ₐₜₚ₎ channel. Specific compounds which are suitable as potassium K₍ₐₜₚ₎ channel openers for the purposes described herein may be selected from the group consisting of pinacidil; cromakalim; diazoxide; BPDZ 44; BPDZ 49; BPDZ 62; BPDZ 73; BPDZ 79; BPDZ 83; BPDZ 109; BPDZ 154; BPDZ 216 (=NNC 55-9216); NN414 (all: see e.g. Hansen et al.); NNC 55-0118 (see e.g. T. M. Tagmose et al., J. Med. Chem. 47 (2004) 3202-3211); NNC 55-0462 (see e.g. Hansen et al.), MCC-134 (see e.g. M. J. Coghlan et al., J. Med. Chem. 44 (2001) 1627-1653); Iosimendan; SR 47063 and WAY 135201.

Description of the Pharmacological Test Methods

1. Electrophysiological Examination of Test Compounds on the K_{V1.3}-Mediated Potassium Current

Methods

Molecular Biology

cDNA coding for the human potassium K_{v1.3} was cloned into a standard vector. A C-terminal epitope-tag was introduced via PCR. The plasmid was sequenced and subsequently introduced into cells and a clonal cell line was established. Expression of protein was analyzed by means of immunofluorescence using antibodies directed against the epitope-tag. The electrophysiological investigations have shown no difference of the biophysical properties of the tagged potassium K_{v1.3} channel versus the un-tagged form.

Cell Culture

Experiments were performed in CHO cells stably expressing the potassium K_{v1.3} potassium channel. Cells were grown at 37° C. and 5% CO₂ in 25 ml flasks in 6 ml MEM ALPHA Medium supplemented with 10% (v/v) heat inactivated fetal calf serum, 1% (v/v) P/S/G-solution and the appropriate selection marker.

Experimental Procedure

Patch-clamp experiments were performed in the voltage-clamp mode (Hamill et al., 1981) and whole-cell currents were recorded. Patch pipettes were pulled from borosilicate glass tubes. Current signals were amplified and digitized by an EPC patch-clamp amplifier (HEKA-Electronics, Lambrecht, Germany), stored and analyzed on a personal computer using the Pulse/Pulsefit software (HEKA, Lambrecht, Germany). Experiments were conducted at room temperature.

Stimulation protocol for the potassium K_{v1.3}-mediated current.

For investigating effects and reversibility of a test compound on the K_{v1.3} potassium channel, CHO cells were clamped at a holding potential (HP) of −80 mV. The following stimulation protocol (scheme 1) was applied successively and the induced currents have been recorded:

Duration of the +40 mV pulse was 1000 ms, and pulse cycling rate was 1/10 s (0.1 Hz) to investigate compound effect.

Compound Application Protocol

The application protocol of the test compound is depicted in scheme 2. The first 14 stimuli were required to achieve steady state of the current amplitude. Nonspecific current reduction was calculated and serves for correction procedures during data analysis. After the 14th stimulus, the test compound was applied into the bath via Teflon and silicone tubings (indicated by an arrow) and was supposed to reach the cell after 6 additional stimuli. The perfusion was validated by using a defined drop rate of 10 drops per 10⁻¹² s. Effect of the test compound was analyzed between stimulus nos. 20 and 50 (ca. 5 min.) and time of wash-out between stimulus nos. 51 and 80 (5 min.). Start of test compound application and of wash-out is indicated by arrows. Number of stimuli of each single episode is shown in the protocol of scheme 2.

Data Compilation

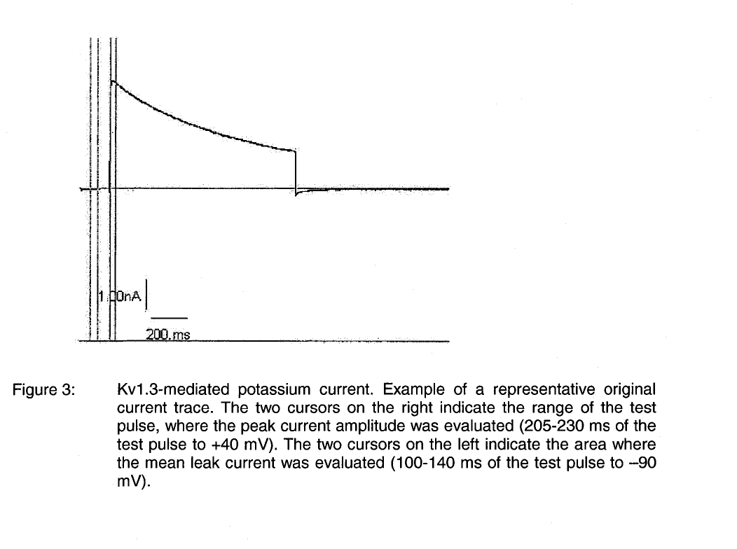

The appropriate experiment was selected from the data tree in the Pulse software. The sampled pulses in this sequence were played back and displayed on the oscilloscope screen. The leak current amplitude during the prepulse to −90 mV and the peak current amplitude of the test-pulse to +40 mV were measured by placing the cursors on the oscilloscope screen (FIG. 3). The values were automatically written and saved to a notebook in the Pulse software. The data from this notebook were imported into Excel for further analysis. The graphical presentation, the evaluation of run-down correction and compound effect of each experiment were performed in SigmaPlot by copying the results from Excel.

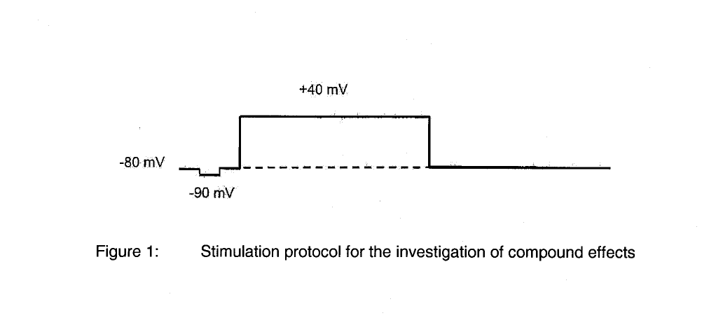

FIG. 1: K_{v1.3}-mediated potassium current. Example of a representative original current trace. The two cursors on the right indicate the range of the test pulse, where the peak current amplitude was evaluated (205-230 ms of the test pulse to +40 mV). The two cursors on the left indicate the area where the mean leak current was evaluated (100-140 ms of the test pulse to −90 mV).

Data analysis for potassium K_{v1.3}-mediated currents

The amplitude of the potassium K_{v1.3}-mediated current gradually decreases over time in some experiments, even in control conditions (called “run-down”). In order to accurately quantify the extent of block, the time course of the current amplitude during the initial period (first 20 stimuli) of the experiment was calculated by a biexponential fit of the equation:
Y=a*exp(−cx)+b*exp(−dx) (1)

a, b, c and d were calculated by the fitting routine in Excel or SigmaPlot.

The fit was extrapolated to the complete time of compound application and washout period. The value for the amplitude under control condition was given by the curve fit (curvexy value).

For the evaluation of compound effect, the curve value under control condition (l curve50) and the current amplitude during test item application (l drug50) were used.

The current reduction was calculated according to the following equation:
relative remaining current=(l drug50/l curve50) (2)

The current recovery was calculated according to the following equation:
relative current recovery=(l wash80/l curve80) (3)

Data are presented as mean±S.D. (standard deviation).

Concentration/response relations were calculated by non-linear least-squares fits of the equation
l/lmax=1/(1+(C/IC50)nH) (4)

to the individual data points. The Hill coefficient (nH) and the half-maximal inhibiting concentration (IC50) were calculated by the fitting routine of the SigmaPlot software.

Results

Effects on the potassium K_{v1.3}-mediated current. The following chemical compound, hereinafter referred to as example compound 1, has been investigated:

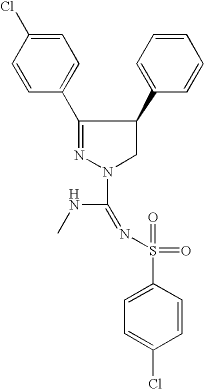

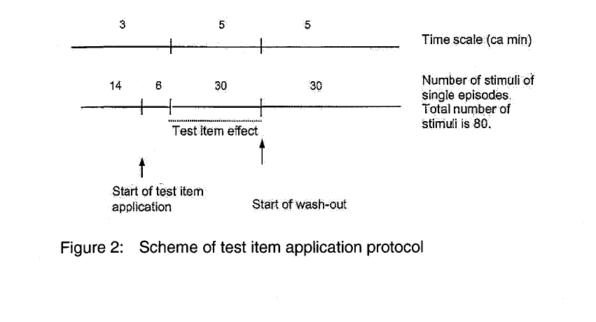

In presence of example compound 1, the outward current amplitudes were reduced in a concentration-dependent manner, demonstrating an effect of example compound 1 on the K_{v1.3}-mediated potassium current. For concentrations of 10 μM and 30 μM, the same effect for example compound 1 was measured in the presence of 0.1% Bovine Serum Albumine (BSA) to overcome the low solubility of example compound 1. In FIG. 2B, a typical example for the time course upon application of 10 μM example compound 1 is depicted, showing a significant current reduction of the initial amplitude.

FIG. 3 shows the concentration-response relationship for the block of the potassium K_{v1.3} channel by example compound 1. Equation (4) was fitted to the data points. The apparent IC50 in the presence of 0.1% BSA was 10.3±3.7 μM, and nH was 0.72±0.22. The extrapolated curve fit for concentrations higher than 10 μM is shown as dashed line: due to limited solubility, it could not be determined if a block of >50-60% may be achieved and therefore the IC50 value should be considered as an estimate.

Legend to table 1: Effects of example compound 1 on potassium K_{v1.3} in presence and absence of 1, 3, 10 and 30 μM in absence or presence of 0.1% BSA. In the first column the corresponding experiments are listed. Current amplitudes in columns 2-5 represent the run-down and leak current corrected steady-state amplitudes measured at +40 mV.

TABLE 1
Potassium K_{V1.3}-mediated current amplitude in
absence and presence of example compound 1
Current amplitude (pA)

| | | 1 μM^{a} | | 3 μM^{a} | | 10 μM^{a} | |
|---|---|---|---|---|---|---|---|
| | | example | | example | | example | |
| 1 μM^{a} | | compound | | compound | | compound 1 | |
| example | | 1 (0.1% | | 1 (0.1% | | (0.1% | |
| compound 1 | Wash^{b} | BSA) | Wash^{b} | BSA) | Wash^{b} | BSA) | Wash^{b} |
| 0.72 ± 0.19 | 0.49 ± 0.19 | 0.93 ± 0.07 | 1.27 ± 0.29 | 0.61 ± 0.06 | 0.53 ± 0.01 | 0.55 ± 0.07 | 0.74 ± 0.20 |

^{a}Relative remaining current amplitude calculated according to: I drug50/I curve50
^{b}The relative remaining current amplitude reflects the reversibility of test item effect after wash-period. It was calculated according to: I wash80/I curve80.

2. Electrophysiological Examination of Test Compounds on the K_{v1.3}-Mediated Potassium Current (Potassium K_{v1.3} Autopatch Electrophysiology Method)

In another set of experiments, the inhibition of the potassium K_{v1.3} channel has been measured. For purposes herein, it has been previously stated that a preferred potassium K_{v1.3} channel inhibitor inhibits the potassium K_{v1.3} channel greater than about 40%, preferably greater than about 60%, more preferably greater than about 80%, even more preferably greater than about 90% and most preferably greater than about 95% or above.

Cells stably transfected with cDNA for human potassium K_{v1.3} (in pcDNA3.1) were grown in Ex-cell 302 serum-free medium for CHO cells, supplemented with 10 μl/ml [100×] glutamine, 500 μg/ml G418, and 1% HT supplement (50×, hypoxanthine and thymidine). The cells were cultured in 350 ml spinner (Techne) spun at 80 rpm, at 37° C. in a water-saturated 5% CO₂ incubator. The day of the preparation an aliquot of cells was 5 times diluted in fresh media and counted with a glass counting chamber type Malassez. Then six tubes of 10 ml were prepared at 6×10⁶ c/ml. The tubes were then placed at 4° C. until their use.

The external bathing solution contained (in mM): 150 NaCl, 10 KCl, 1 MgCl₂, 3 CaCl₂ and 10 HEPES. The pH was adjusted to 7.4 with NaOH. Patch pipettes were filled with a pipette solution of composition (in mM): 100 K-Gluconate, 20 KCl, 1 MgCl₂, 1 CaCl₂, 10 HEPES, 11 EGTA, 5 ATP-Na₂ and 2 Glutathione. The pH was adjusted to 7.2 with KOH. Using fresh 100 ml bathing solution, 0.05% BSA re-suspension solution (0.05 g BSA/100 ml bather) was prepared.

Compounds were dissolved in DMSO (100%) and made up in the external bather at concentrations of 1 μM and 10 μM. All experiments were conducted at room temperature.

Two tubes of cell suspension were centrifuged at 1000 rpm for 4 minutes at room temperature. 10 ml of supernatant was carefully aspirated and discarded from each tube, with care being taken not to aspirate the cell pellet sitting at the bottom of the tube. The cell pellet of each tube was broken-up by gentle manual agitation of the tube. 600 μl of re-suspension solution was added to the cell pellet, followed by a gentle trituration step to re-suspend the cells. Once re-suspended, 600 μl of solution per tube was removed from each tube, such that 1.2 ml of cell suspension was obtained in total and placed in a temperature-controlled cell hotel, set at either 4° C. or dew point. Cells were maintained in suspension in the hotel through gentle trituration every 30 s via an automatic cell suspension system.

Harvard borosilicate capillary glass (GC1507F-10, 1.5 mm ID×1.17 mm OD, supplied in 100 mm lengths) was cut down to 24 mm lengths using a Dagen capillary cutter. Short (13 mm) patch pipettes were pulled using a 2-stage pull on a specially adapted DMZ pipette puller (Zeitz Instruments). Patch pipettes typically have resistances of 2.3-3.5 MΩ. As the patch pipettes were pulled in batches, pipette tip resistance was measured every tenth pipette using a Tenma meter in order to maintain consistency of tip resistance. Pulled pipettes were stored in Petri dish before filling.

Each pipette was fully back-filled (from the tip to the end) using the internal pipette solution and the pipette tip was dipped in Sigmacote (Sigma). The pipette was then precisely inserted, tip first, into a pipette holder using a custom-made, pneumatic insertion rig, with the blunt end of the patch pipette sitting proud of the bottom of the pipette holder.

Whole cell patch-clamp recordings (WCRs) were made using the AP2, which incorporates an EPC9 or EPC10 amplifier (HEKA, Germany) under control of Pulse software (v8.54 or v8.76, HEKA, Germany), the patch plate contact fixture, a Gilson autosampler for cell delivery (the cell sampler), a Gilson autosampler for drug preparation (the auto-sampler), the drug application system (DAS), a feedback-controlled suction device for enabling high resistance GΩ seals to be formed for the whole cell recording mode to be attained, a cell re-suspension system, a temperature-controlled cell hotel, a vacuum line and associated pumps for applying suction and for draining the patch plate wells of bather.

Under control of the AP2 software, automated patch-clamping was initiated. A Gilson sampler needle visited the cell hotel and takes up 15 μl of cell suspension. The sampler needle then visited the first designated patch pipette in the patch plate. The sampler needle slowly descended towards the patch pipette until the liquid interface was detected. The presence of a cell at the pipette tip was detected by the resistance of the tip exceeding 50 MΩ. Once a cell was detected, suction was applied to the pipette to obtain a GΩ seal. Once a GΩ seal was obtained and has stabilized for 60 s (at 0 mmHg suction), suction was again applied in ramps to enable membrane break-in and the gaining of the WCR configuration. During application of the suction ramp, the membrane holding voltage (Vₘₑₘ) was hyperpolarized in 10 mV steps until the experimental holding voltage (V_{hold}) was obtained.

Qualification stages prior to perfusion and drug application ensure that the observed potassium K_{v1.3} current met the user-determined criteria for the experiment. Only those cells with an I_{K}>400 pA were used. Cells were continuously perfused with external solution at a flow rate of 1.8-2 ml/minute. The perfusion chamber has a working volume 100 μl and allows for rapid exchange of drug solutions.

Once the qualification criteria have been met, test compound was applied to the cell via the DAS system. Compound at a stock concentration was held in a 96 well plate on the autosampler. 80 μl of compound was drawn up by the autosampler and diluted with bather to the requisite concentration by the autosampler. The degree of dilution of each compound required was automatically determined by Autopatch.exe according to the final concentration required to be applied to the cell. Each compound was applied for 5 minutes, following which the compound was washed out by bather. Recovery, or otherwise, of the potassium K_{v1.3} response was monitored by Autopatch.exe, such that a second compound was applied only when the potassium K_{v1.3} current has returned to the pre-drug application amplitude within a given time period. Should recovery not be sufficient during this period, Autopatch.exe terminates the experiment and moves on to the next recording site. Online analysis of the potassium K_{v1.3} current during the application of compounds was performed by the Autopatch software.

Electrophysiology voltage-step protocols and analysis of data was performed as follows. Data was sampled at 5 kHz, and filtered with a −3 dB bandwidth of 2.5 kHz. Cells were held at a voltage of −80 mV. Currents were applied as a voltage step of +30 mV for 500 ms in duration every 10 s. Total charge was measured during the whole of voltage step by AP2 and plotted by the APGraph.exe software.

During potassium K_{v1.3} experiments charge was measured as the integral of the current with respect to time during 1-99% (5-495 ms) of the 500 ms step to +30 mV in the absence (Q_{Control}) and presence (Q_{Drug}) of drug. Control potassium K_{v1.3} charge measurements were taken as the average of the two sweeps immediately prior to drug addition. Percentage Charge inhibition was calculated as shown in

$$\begin{matrix}Equation 1 & \\ \% Charge Inhibition = ( 1 - \frac{{Q}_{Drug}}{{Q}_{Control}} ) \times 100 & ( 1 )\end{matrix}$$

Equation 1—Percentage Charge Inhibition wherein Q_{Control} and Q_{Drug} are the charge measured prior to and following equilibration in drug, respectively.

| | | Inhibition |
|---|---|---|
| | | of the |
| Research Code/ | | Potassium K_{v1.3} |
| Trade Name | Structural formulae | channel (%) |
| | 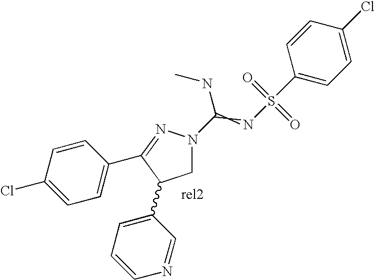 | 88.7 |
| | 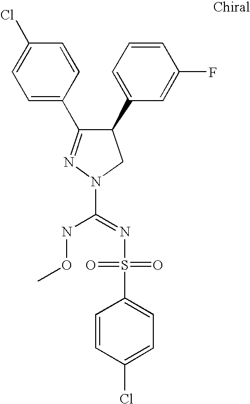 | 76.0 |
| | 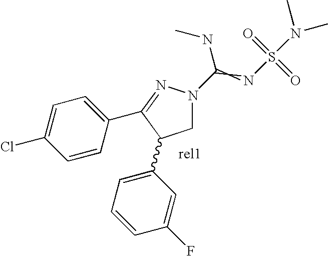 | 88.1 |
| | 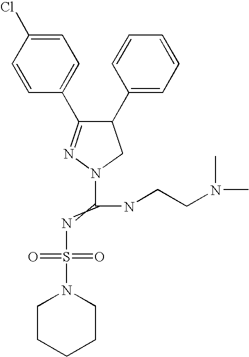 | 58.4 |
| | 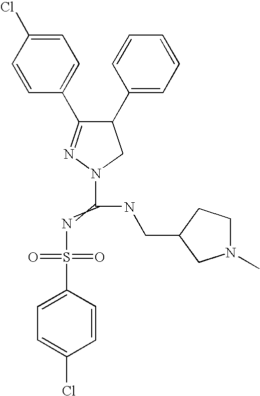 | 96.2 |
| | 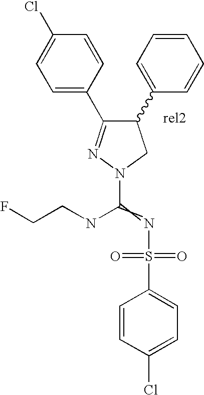 | 77.1 |
| | 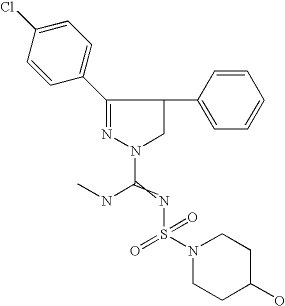 | 92.8 |
| | 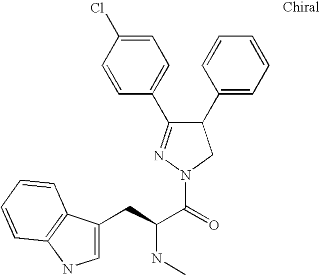 | 87.8 |
| HU-308 | 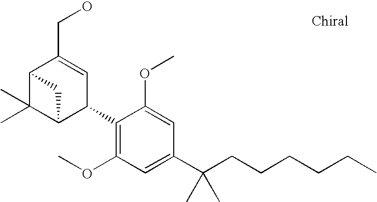 | 81.2 |
| | 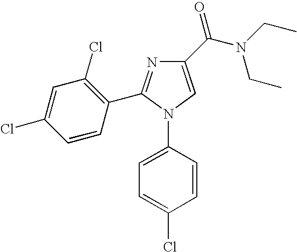 | 76.2 |
| Diazoxide | 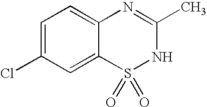 | 46.5 |
| NN414 | 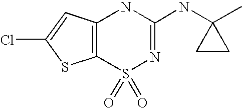 | 45.1 |
| R(+)-WIN55212-2 | 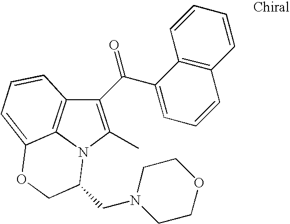 | 81.5 |
| Rimonabant | 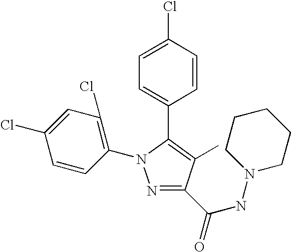 | 89.2 |
| SR-147778 | 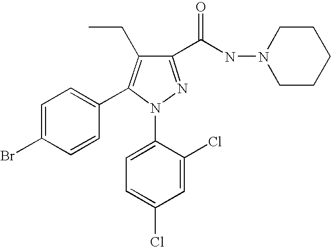 | 63.7 |

The potassium K_{v1.3} channel inhibitors described herein and/or the compounds having potassium K_{v1.3} channel inhibiting properties and also either or both CBₓ modulating properties and/or potassium K₍ₐₜₚ₎ channel opening properties, either alone or in combination with an effective amount of at least one CB₁ antagonist and/or an effective amount of at least one potassium K₍ₐₜₚ₎ channel opener, may be administered in conventional pharmaceutical preparations. The doses to be used may vary individually and will naturally vary according to the type of condition to be treated and the substance used. In general, however, medicinal forms with an active substance content of about 0.2 to about 500 mg, such as, e.g., about 0.2, about 0.4, about 0.6, about 0.8, about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 225, about 250, about 275, about 300, about 325, about 350, about 375, about 400, about 425, about 450, about 475, or about 500 mg, in particular from about 1 to about 200 mg, such as, e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, or about 200, active substance per individual dose are suitable for administration to humans and larger mammals. The potassium K_{v1.3} channel inhibitors described herein and/or the compounds having both potassium K_{v1.3} channel inhibiting properties and also either or both CBₓ modulating properties and/or potassium K₍ₐₜₚ₎ channel opening properties, either alone or in combination with an effective amount of at least one CB₁ antagonist and/or an effective amount of at least one potassium K₍ₐₜₚ₎ channel opener, may be contained for the purposes described herein, together with conventional pharmaceutical auxiliaries and/or carriers, in solid or liquid pharmaceutical preparations. Examples of solid preparations are preparations which can be administered orally, such as tablets, coated tablets, capsules, powders or granules, or alternatively suppositories. These preparations may contain conventional pharmaceutical inorganic and/or organic carriers, such as talcum, lactose or starch, in addition to conventional pharmaceutical auxiliaries, for example lubricants or tablet disintegrating agents. Liquid preparations such as suspensions or emulsions of the potassium K_{v1.3} channel inhibitors described herein and/or the compounds having both potassium K_{v1.3} channel inhibiting properties and also either or both CBₓ modulating properties and/or potassium K₍ₐₜₚ₎ channel opening properties, either alone or in combination with an effective amount of at least one CB₁ antagonist and/or an effective amount of at least one potassium K₍ₐₜₚ₎ channel opener contain the usual diluents such as water, oils and/or suspension agents such as polyethylene glycols and the like. Other auxiliaries may additionally be added, such as preservatives, taste masking agents and the like.

The potassium K_{v1.3} channel inhibitors described herein and/or the compounds having both potassium K_{v1.3} channel inhibiting properties and also either or both CBₓ modulating properties and/or potassium K₍ₐₜₚ₎ channel opening properties, either alone or in combination with an effective amount of at least one CB₁ antagonist and/or an effective amount of at least one potassium K₍ₐₜₚ₎ channel opener may be mixed and formulated with the pharmaceutical auxiliaries and/or carriers. For the production of solid medicament forms, the potassium K_{v1.3} channel inhibitors described herein and/or the compounds having both potassium K_{v1.3} channel inhibiting properties and also either or both CBₓ modulating properties and/or potassium K₍ₐₜₚ₎ channel opening properties, either alone or in combination with an effective amount of at least one CB₁ antagonist and/or an effective amount of at least one potassium K₍ₐₜₚ₎ channel opener may for example be mixed with the auxiliaries and/or carriers in conventional manner and may be wet or dry granulated. The granules or powder can be poured directly into capsules or be pressed into tablet cores in conventional manner. These may be coated in known manner if desired.

All references, including publications, patent applications, and patents, cited herein are hereby incorporated by reference to the same extent as if each reference there individually and specifically indicated to be incorporated by reference were set forth in its entirety herein.

The use of the terms “a” and “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately” unless clearly indicated otherwise by context. Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the claimed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately”. Thus, recitation of ranges of values herein are merely intended to serve as a shorthand method of referring individually to each separate value falling within the range, unless otherwise indicated herein, and each separate value is incorporated into the specification as if it were individually recited herein.

We claim:

1. A method of treating, preventing or providing a medical condition in a subject comprising the steps of:
a. selecting one or more potassium K_{v1.3} channel inhibiting compounds wherein the one or more compounds optionally have one or more additional properties selected from the group consisting of:
i. CB₁ antagonist properties;
ii. CB₁ agonists properties;
iii. CB₂ agonists properties; and
iv. potassium K₍ₐₜₚ₎ channel opening properties.
b. combining the one or more compounds with one or more pharmaceutically acceptable excipients to create a dosage form suitable for administration to a subject; and
c. administering the dosage form to the subject;
wherein the medical condition is selected from the group consisting of: obesity, diabetes mellitus, metabolic syndrome, syndrome X, insulinoma, familial hyperinsulemic hypoglycemia, male pattern baldness, detrusor hyperreactivity, asthma, insufficient glucose metabolism, insulin resistance, hyperglycaemea, glucose intolerance, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain and impotence.

2. The method according to claim 1 wherein the medical condition is selected from the group consisting of: obese type I diabetes, obese type II diabetes, non-obese-type I diabetes, non-obese-type II diabetes and related conditions.

3. The method according to claim 2 wherein the related condition is selected from the group consisting of: insufficient glucose metabolism, insulin resistance, hyperglycaemea and glucose intolerance.

4. The method according to claim 1 wherein the potassium K_{v1.3} channel inhibitor is selected from the group consisting of:
a.) 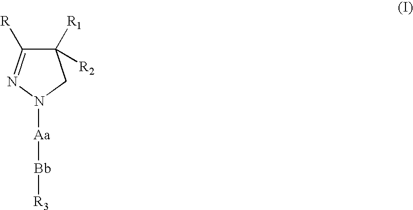
wherein:
R and R₁ are independently selected from the group consisting of: napthyl, phenyl, thienyl and pyridyl wherein phenyl, thienyl and pyridyl may be substituted with 1, 2 or 3 substitutents Y;
R₂ is selected from the group consisting of: hydrogen, hydroxy, C₁₋₃-alkoxy, acetyloxy and propionyloxy;
R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
Aa is selected from the group consisting of: substitutents of formulae (i), (ii), (iii), (iv), (v) and (vi) 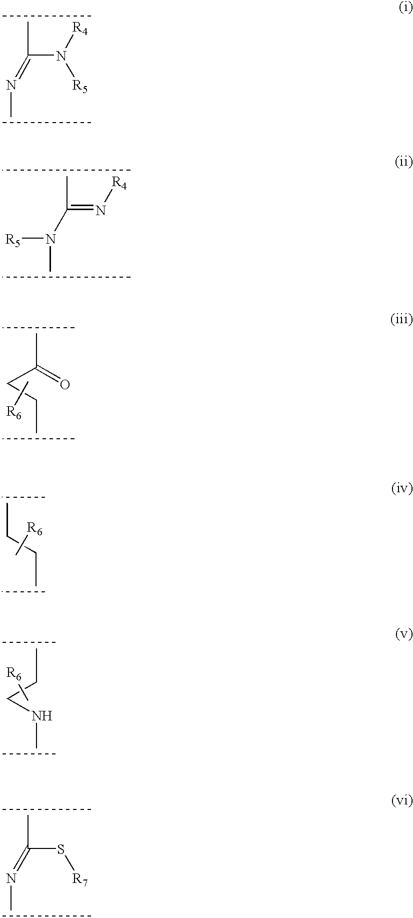
Bb is selected elected from the group consisting of: sulfonyl and carbonyl;
each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
R₄ is selected from the group consisting of: hydrogen, C₁₋₈ branched or unbranched alkyl and C₃₋₈ cycloalkyl; or R₄ is selected from the group consisting of: acetamido, dimethylamino, 2,2,2-trifluoroethyl, phenyl and pyridyl with the proviso that R₅ is hydrogen,
wherein such C₁₋₈ branched or unbranched alkyl and/or C₃₋₈ cycloalkyl alkyl group may be substituted with a hydroxyl group;
R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
wherein such C₁₋₈ branched or unbranched alkyl and/or C₃₋₈ cycloalkyl group may be substituted with hydroxyl and/or fluoro,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₁₃ alkyl, hydroxy, amino, monoalkylamino, dialkylamino or fluoro,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
wherein such phenyl, benzyl, pyridyl, thienyl, pyridylmethyl or phenethyl group may be substituted with 1, 2 or 3 of the substitutents Y,
wherein such monocyclic or bicyclic heterocyclic moiety having 4 to 10 ring atoms may contain one or more heteroatoms selected from the group consisting of: O, N and S,
wherein such monocyclic or bicyclic heterocyclic moiety having 4 to 10 ring atoms may contain a keto or —SO₂— group,
wherein such monocyclic or bicyclic heterocyclic moiety having 4 to 10 ring atoms may be substituted with a CO₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
R₆ is selected from the group consisting of: hydrogen and C₁₋₃ unbranched alkyl;
R₇ is C₁₋₃ unbranched alkyl;
R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
wherein such saturated or unsaturated heterocyclic moiety having 4 to 8 ring atoms may contain an additional heteroatom selected from the group consisting of: N, O and S or may contain a group selected from the group consisting of: a keto or —SO₂— group,
wherein such saturated or unsaturated heterocyclic moiety having 4 to 8 ring atoms may be substituted with C₁₋₄ alkyl;
R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁—together with the nitrogen atom to which they are bonded—form a monocyclic, bicyclic or tricyclic alkyl or alkenyl group,
wherein such branched or unbranched C₁₋₈ alkyl and/or branched or unbranched C₁₋₈ alkenyl groups may contain one or more heteroatoms selected from the group consisting of: O, N, and S,
wherein such branched or unbranched C₁₋₈ alkyl and/or branched or unbranched C₁₋₈ alkenyl groups may contain a group selected from the group consisting of: keto and —SO₂-group and wherein such keto and —SO₂-group may be substituted with a hydroxy or amino group,
wherein such C₃₋₈ cycloalkyl and/or C₃₋₈ cycloalkenyl group may contain one or more ring heteroatoms selected from the group consisting of: O, N and S,
wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
wherein such monocyclic, bicyclic or tricyclic alkyl or alkenyl group may contain ring heteroatoms selected from the group consisting of: O, N and S,
wherein such monocyclic, bicyclic or tricyclic alkyl or alkenyl group may contain a group selected from the group consisting of: keto and SO₂,
wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 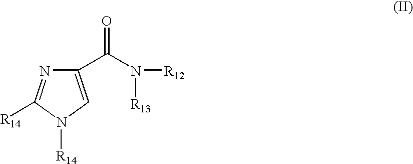
wherein
R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 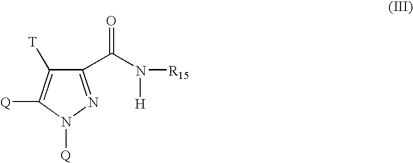
wherein
Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
T is selected from the group consisting of: hydrogen, C₁₋₃ alkyl and C₃₋₆ cycloalkyl which may contain from 1 to 3 heteroatoms selected from the group consisting of: N, O and S;
R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and
a prodrug thereof, a tautomer thereof or a pharmaceutically acceptable salt thereof; and
e.) mixtures of any of the foregoing.

5. The method according to claim 4 wherein R₂ is hydrogen and wherein the 4-position of the 4,5 dihydro pyrazole ring is in the S-configuration.

6. The method according to claim 1 wherein the potassium K_{v1.3} channel inhibitor is selected from the group consisting of: 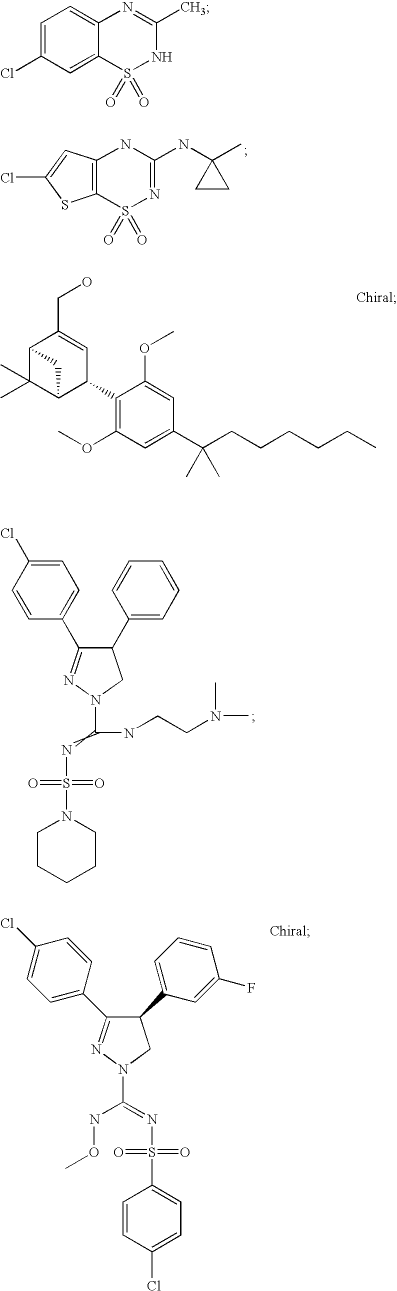 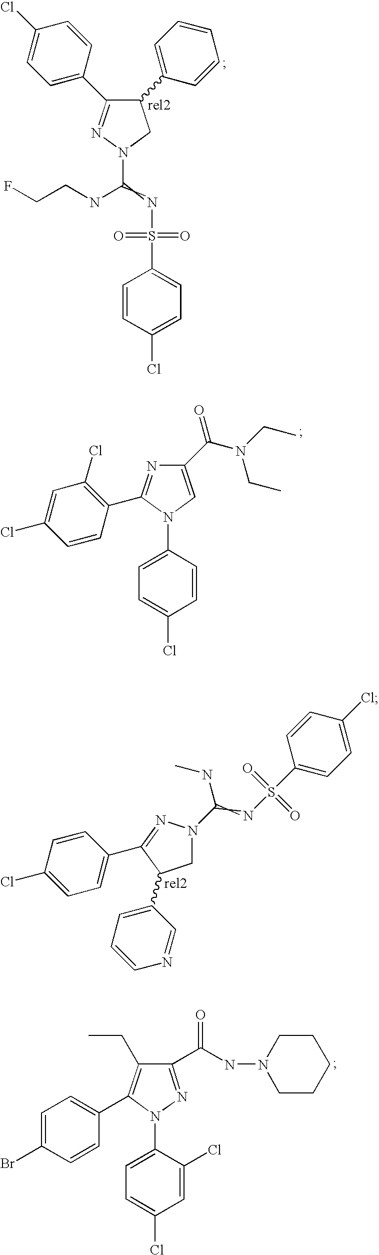 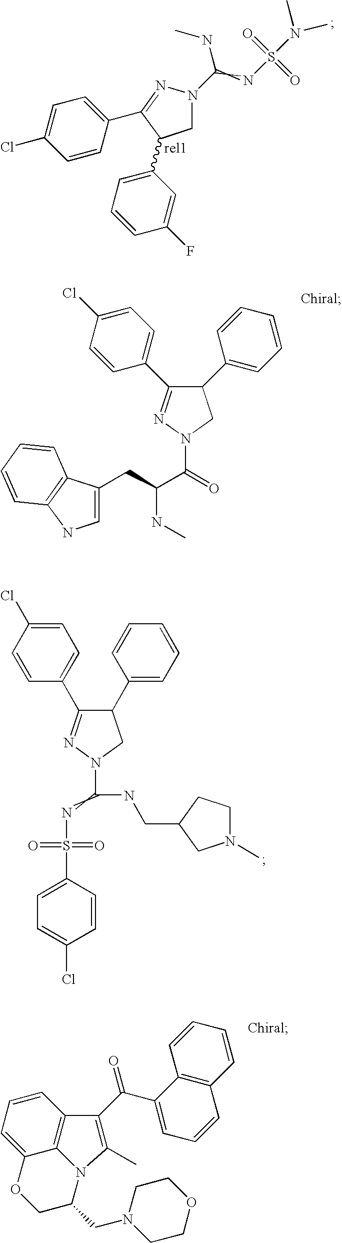 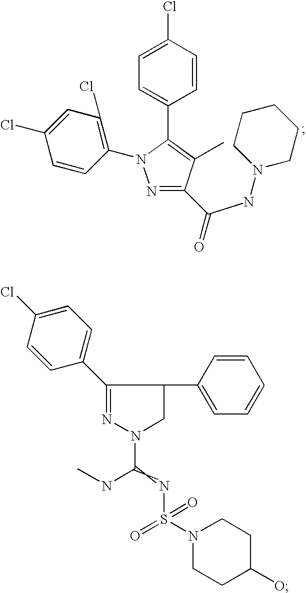
and mixtures thereof.

7. A pharmaceutical composition comprising a pharmacologically effective amount of at least one potassium K_{v1.3} channel inhibitor wherein the one or more compounds optionally have one or more additional properties selected from the group consisting of:
i. CB₁ antagonist properties;
ii. CB₁ agonists properties;
iii. CB₂ agonists properties; and
iv. potassium K₍ₐₚₜ₎ channel opening properties.
wherein the pharmaceutical composition is for treating, preventing or providing a medical condition selected from the group consisting of: obesity, diabetes mellitus, metabolic syndrome, syndrome X, insulinoma, familial hyperinsulemic hypoglycemia, male pattern baldness, detrusor hyperreactivity, asthma, insufficient glucose metabolism, insulin resistance, hyperglycaemea, glucose intolerance, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain and impotence.

8. The pharmaceutical composition according to claim 7 wherein the medical condition is selected from the group consisting of: obese type I diabetes, obese type II diabetes, non-obese-type I diabetes, non-obese-type II diabetes and related conditions.

9. The pharmaceutical composition according to claim 8 wherein the related condition is selected from the group consisting of: insufficient glucose metabolism, insulin resistance, hyperglycaemea and glucose intolerance.

10. The pharmaceutical composition according to claim 7 wherein the potassium K_{v1.3} channel inhibitor is selected from the group consisting of:
a.) 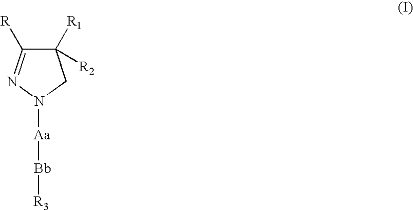
wherein:
R and R₁ are independently selected from the group consisting of: napthyl, phenyl, thienyl and pyridyl wherein phenyl, thienyl and pyridyl may be substituted with 1, 2 or 3 substitutents Y;
R₂ is selected from the group consisting of: hydrogen, hydroxy, C₁₋₃-alkoxy, acetyloxy and propionyloxy;
R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
Aa is selected from the group consisting of: substitutents of formulae (i), (ii), (iii), (iv), (v) and (vi) 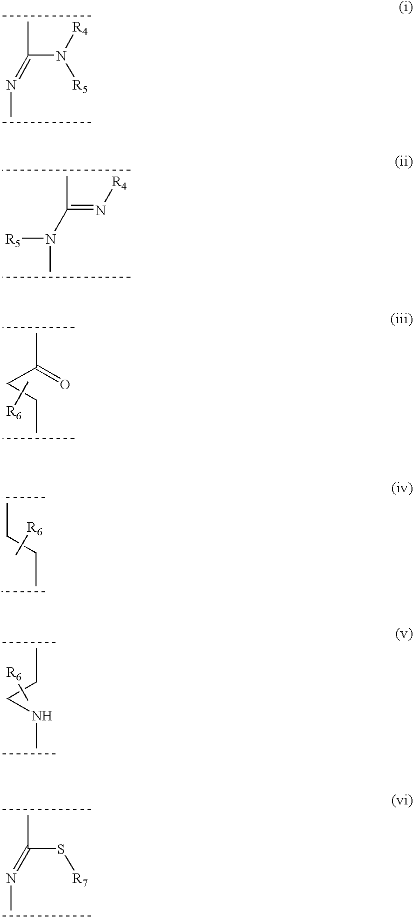
Bb is selected elected from the group consisting of: sulfonyl and carbonyl;
each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
R₄ is selected from the group consisting of: hydrogen, C₁₋₈ branched or unbranched alkyl and C₃₋₈ cycloalkyl; or R₄ is selected from the group consisting of: acetamido, dimethylamino, 2,2,2-trifluoroethyl, phenyl and pyridyl with the proviso that R₅ is hydrogen,
wherein such C₁₋₈ branched or unbranched alkyl and/or C₃₋₈ cycloalkyl alkyl group may be substituted with a hydroxyl group;
R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
wherein such C₁₋₈ branched or unbranched alkyl and/or C₃₋₈ cycloalkyl group may be substituted with hydroxyl and/or fluoro,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₃ alkyl, hydroxy, amino, monoalkylamino, dialkylamino or fluoro,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
wherein such phenyl, benzyl, pyridyl, thienyl, pyridylmethyl or phenethyl group may be substituted with 1, 2 or 3 of the substitutents Y,
wherein such monocyclic or bicyclic heterocyclic moiety having 4 to 10 ring atoms may contain one or more heteroatoms selected from the group consisting of: O, N and S,
wherein such monocyclic or bicyclic heterocyclic moiety having 4 to 10 ring atoms may contain a keto or —SO₂— group,
wherein such monocyclic or bicyclic heterocyclic moiety having 4 to 10 ring atoms may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
R₆ is selected from the group consisting of: hydrogen and C₁₋₃ unbranched alkyl;
R₇ is C₁₋₃ unbranched alkyl;
R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
wherein such saturated or unsaturated heterocyclic moiety having 4 to 8 ring atoms may contain an additional heteroatom selected from the group consisting of: N, O and S or may contain a group selected from the group consisting of: keto or —SO₂-group,
wherein such saturated or unsaturated heterocyclic moiety having 4 to 8 ring atoms may be substituted with C₁₋₄ alkyl;
R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁—together with the nitrogen atom to which they are bonded—form a monocyclic, bicyclic or tricyclic alkyl or alkenyl group,
wherein such branched or unbranched C₁₋₈ alkyl and/or branched or unbranched C₁₋₈ alkenyl groups may contain one or more heteroatoms selected from the group consisting of: O, N, and S,
wherein such branched or unbranched C₁₋₈ alkyl and/or branched or unbranched C₁₋₈ alkenyl groups may contain a group selected from the group consisting of: keto and —SO₂— group and wherein such keto and —SO₂-group may be substituted with a hydroxy or amino group,
wherein such C₃₋₈ cycloalkyl and/or C₃₋₈ cycloalkenyl group may contain one or more ring heteroatoms selected from the group consisting of: O, N and S,
wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
wherein such monocyclic, bicyclic or tricyclic alkyl or alkenyl group may contain ring heteroatoms selected from the group consisting of: O, N and S,
wherein such monocyclic, bicyclic or tricyclic alkyl or alkenyl group may contain a group selected from the group consisting of: keto and SO₂,
wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 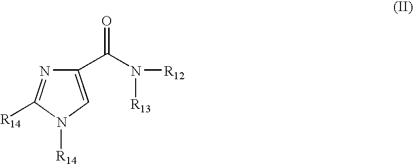
wherein
R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 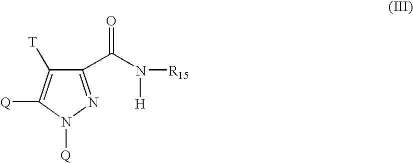
wherein
Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
T is selected from the group consisting of: hydrogen, C₁₋₃ alkyl and C₃₋₆ cycloalkyl which may contain from 1 to 3 heteroatoms selected from the group consisting of: N, O and S;
R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and a prodrug thereof, a tautomer thereof or a pharmaceutically acceptable salt thereof; and
e.) mixtures of any of the foregoing.

11. The pharmaceutical composition according to claim 10 wherein R₂ is hydrogen and wherein the 4-position of the 4,5 dihydro pyrazole ring is in the S-configuration.

12. The pharmaceutical composition according to claim 7 wherein the potassium K_{v1.3} channel inhibitor is selected from the group consisting of: 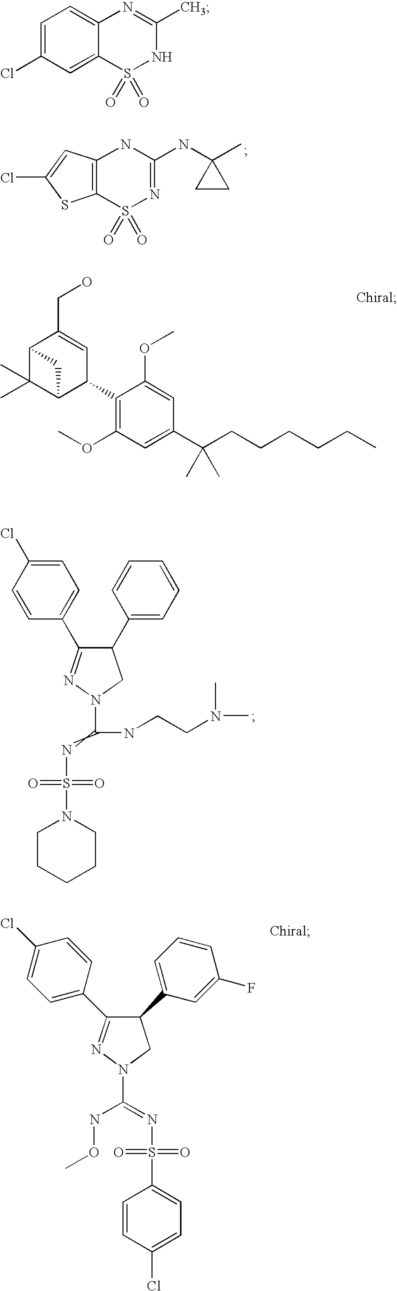 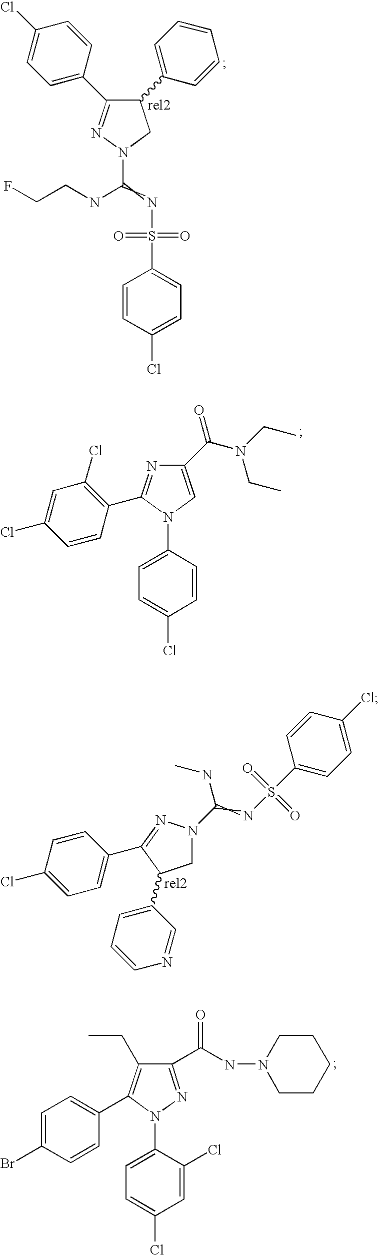 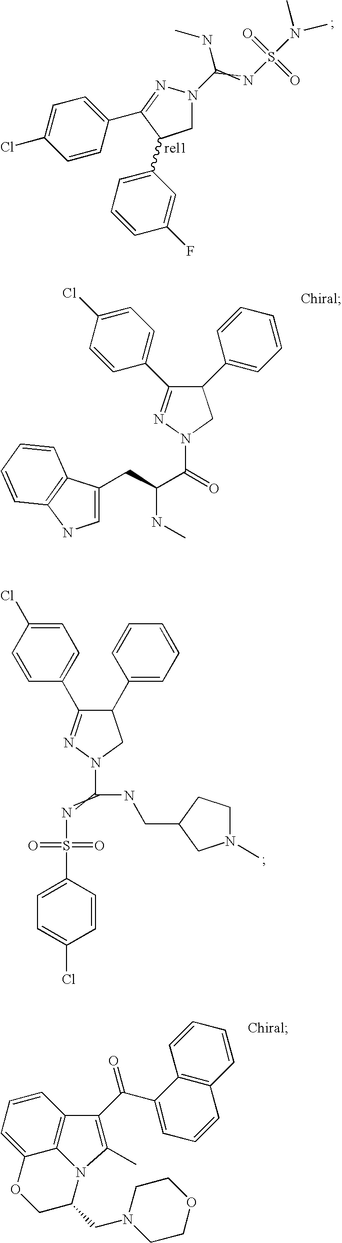 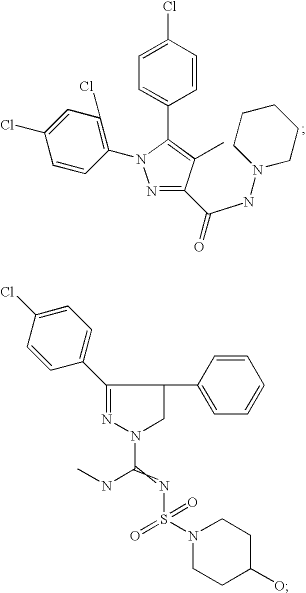
and mixtures thereof.

13. A pharmaceutical composition prepared by a process comprising the steps of:
a. selecting one or more potassium K_{v1.3} channel inhibiting compounds wherein the one or more compounds optionally have one or more additional properties selected from the group consisting of:
i. CB₁ antagonist properties;
ii. CB₁ agonists properties;
iii. CB₂ agonists properties; and
iv. potassium K₍ₐₜₚ₎ channel opening properties.
b. combining the one or more compounds with one or more pharmaceutically acceptable excipients to create a dosage form suitable for administration to a subject;
wherein the pharmaceutical composition is used to treat, prevent or provide a medical condition in a subject, the medical condition is selected from the group consisting of: obesity, diabetes mellitus, metabolic syndrome, syndrome X, insulinoma, familial hyperinsulemic hypoglycemia, male pattern baldness, detrusor hyperreactivity, asthma, insufficient glucose metabolism, insulin resistance, hyperglycaemea, glucose intolerance, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain and impotence.

14. The method according to claim 13 wherein the medical condition is selected from the group consisting of: obese type I diabetes, obese type II diabetes, non-obese-type I diabetes, non-obese-type II diabetes and related conditions.

15. The method according to claim 14 wherein the related condition is selected from the group consisting of: insufficient glucose metabolism, insulin resistance, hyperglycaemea and glucose intolerance.

16. The method according to claim 13 wherein the potassium K_{v1.3} channel inhibitor is selected from the group consisting of:
a.) 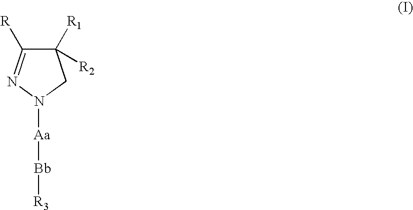
wherein:
R and R₁ are independently selected from the group consisting of: napthyl, phenyl, thienyl and pyridyl wherein phenyl, thienyl and pyridyl may be substituted with 1, 2 or 3 substitutents Y;
R₂ is selected from the group consisting of: hydrogen, hydroxy, C₁₋₃-alkoxy, acetyloxy and propionyloxy;
R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
Aa is selected from the group consisting of: substitutents of formulae (i), (ii), (iii), (iv), (v) and (vi) 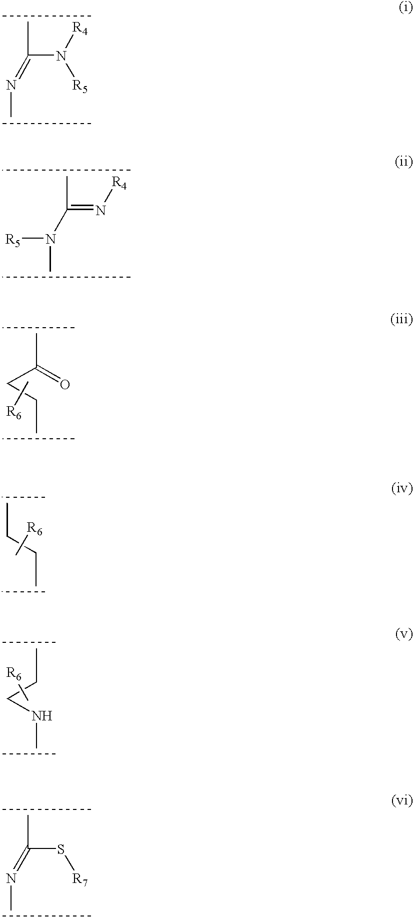
Bb is selected elected from the group consisting of: sulfonyl and carbonyl;
each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
R₄ is selected from the group consisting of: hydrogen, C₁₋₈ branched or unbranched alkyl and C₃₋₈ cycloalkyl; or R₄ is selected from the group consisting of: acetamido, dimethylamino, 2,2,2-trifluoroethyl, phenyl and pyridyl with the proviso that R₅ is hydrogen,
wherein such C₁₋₈ branched or unbranched alkyl and/or C₃₋₈ cycloalkyl alkyl group may be substituted with a hydroxyl group;
R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
wherein such C₁₋₈ branched or unbranched alkyl and/or C₃₋₈ cycloalkyl group may be substituted with hydroxyl and/or fluoro,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₃ alkyl, hydroxy, amino, monoalkylamino, dialkylamino or fluoro,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
wherein such phenyl, benzyl, pyridyl, thienyl, pyridylmethyl or phenethyl group may be substituted with 1, 2 or 3 of the substitutents Y,
wherein such monocyclic or bicyclic heterocyclic moiety having 4 to 10 ring atoms may contain one or more heteroatoms selected from the group consisting of: O, N and S,
wherein such monocyclic or bicyclic heterocyclic moiety having 4 to 10 ring atoms may contain a keto or —SO₂— group,
wherein such monocyclic or bicyclic heterocyclic moiety having 4 to 10 ring atoms may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
R₆ is selected from the group consisting of: hydrogen and C₁₋₃ unbranched alkyl;
R₇ is C₁₋₃ unbranched alkyl;
R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
wherein such saturated or unsaturated heterocyclic moiety having 4 to 8 ring atoms may contain an additional heteroatom selected from the group consisting of: N, O and S or may contain a group selected from the group consisting of: a keto or —SO₂— group,
wherein such saturated or unsaturated heterocyclic moiety having 4 to 8 ring atoms may be substituted with C₁₋₄ alkyl;
R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁-together with the nitrogen atom to which they are bonded—form a monocyclic, bicyclic or tricyclic alkyl or alkenyl group,
wherein such branched or unbranched C₁₋₈ alkyl and/or branched or unbranched C₁₋₈ alkenyl groups may contain one or more heteroatoms selected from the group consisting of: O, N, and S,
wherein such branched or unbranched C₁₋₈ alkyl and/or branched or unbranched C₁₋₈ alkenyl groups may contain a group selected from the group consisting of: keto and —SO₂-group and wherein such keto and —SO₂-group may be substituted with a hydroxy or amino group,
wherein such C₃₋₈ cycloalkyl and/or C₃₋₈cycloalkenyl group may contain one or more ring heteroatoms selected from the group consisting of: O, N and S,
wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
wherein such monocyclic, bicyclic or tricyclic alkyl or alkenyl group may contain ring heteroatoms selected from the group consisting of: O, N and S,
wherein such monocyclic, bicyclic or tricyclic alkyl or alkenyl group may contain a group selected from the group consisting of: keto and SO₂,
wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 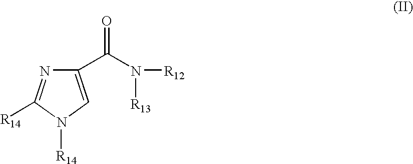
wherein
R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 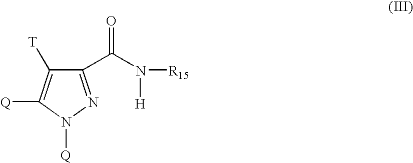
wherein
Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
T is selected from the group consisting of: hydrogen, C₁₋₃ alkyl and C₃₋₆ cycloalkyl which may contain from 1 to 3 heteroatoms selected from the group consisting of: N, O and S;
R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and a prodrug thereof, a tautomer thereof or a pharmaceutically acceptable salt thereof; and
e.) mixtures of any of the foregoing.

17. The method according to claim 16 wherein R₂ is hydrogen and wherein the 4-position of the 4,5 dihydro pyrazole ring is in the S-configuration.

18. The method according to claim 13 wherein the potassium K_{v1.3} channel inhibitor is selected from the group consisting of: 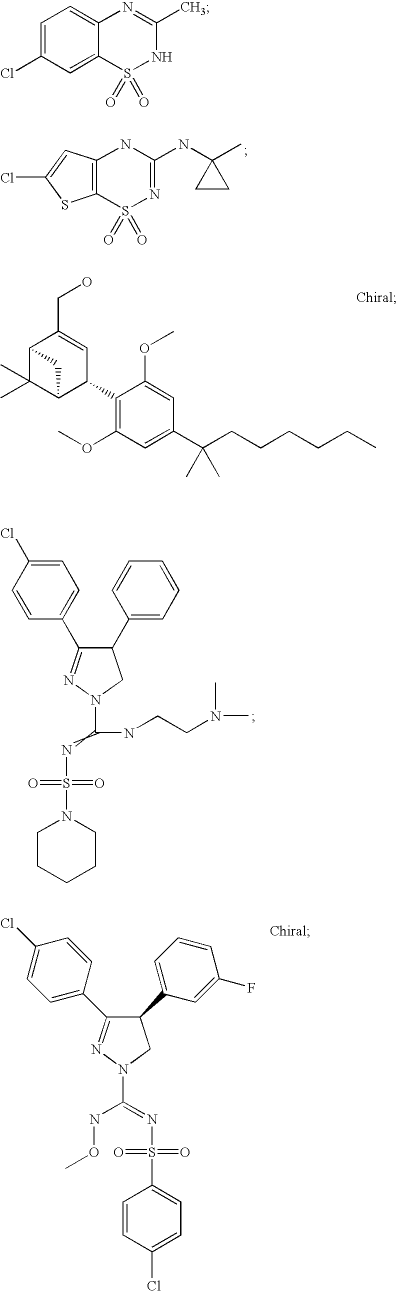 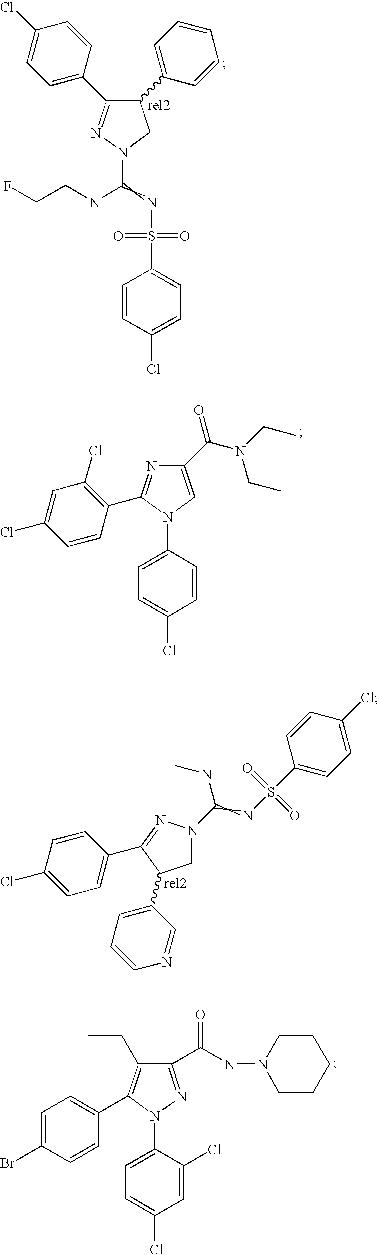 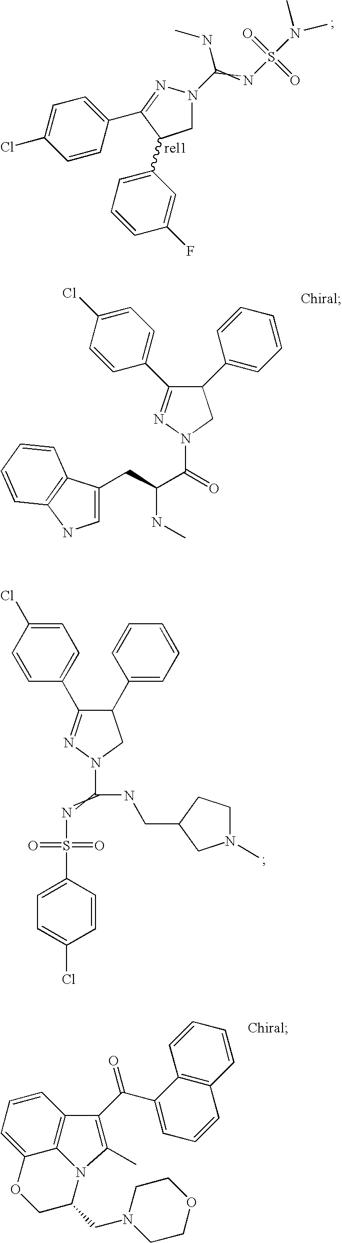 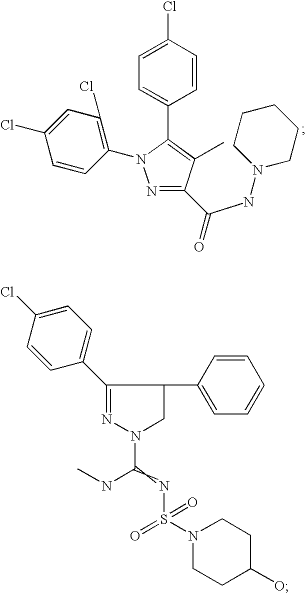
and mixtures thereof.

19. A method of inhibiting a potassium K_{v1.3} channel in a subject comprising the step of administering one or more of the compounds selected from the group consisting of:
a.) 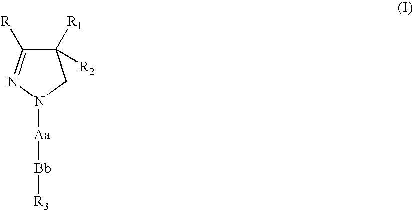
wherein:
R and R₁ are independently selected from the group consisting of: napthyl, phenyl, thienyl and pyridyl wherein phenyl, thienyl and pyridyl may be substituted with 1, 2 or 3 substitutents Y;
R₂ is selected from the group consisting of: hydrogen, hydroxy, C₁₋₃-alkoxy, acetyloxy and propionyloxy;
R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
Aa is selected from the group consisting of: substitutents of formulae (i), (ii), (iii), (iv), (v) and (vi) 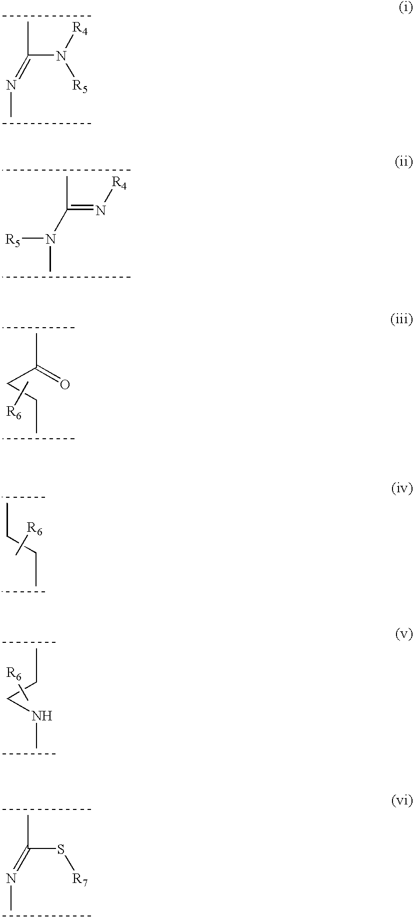
Bb is selected elected from the group consisting of: sulfonyl and carbonyl;
each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
R₄ is selected from the group consisting of: hydrogen, C₁₋₈ branched or unbranched alkyl and C₃₋₈ cycloalkyl; or R₄ is selected from the group consisting of: acetamido, dimethylamino, 2,2,2-trifluoroethyl, phenyl and pyridyl with the proviso that R₅ is hydrogen, wherein such C₁₋₈ branched or unbranched alkyl and/or C₃₋₈ cycloalkyl alkyl group may be substituted with a hydroxyl group;
R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
wherein such C₁₋₈ branched or unbranched alkyl and/or C₃₋₈ cycloalkyl group may be substituted with hydroxyl and/or fluoro,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₃ alkyl, hydroxy, amino, monoalkylamino, dialkylamino or fluoro,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
wherein such phenyl, benzyl, pyridyl, thienyl, pyridylmethyl or phenethyl group may be substituted with 1, 2 or 3 of the substitutents Y,
wherein such monocyclic or bicyclic heterocyclic moiety having 4 to 10 ring atoms may contain one or more heteroatoms selected from the group consisting of: O, N and S,
wherein such monocyclic or bicyclic heterocyclic moiety having 4 to 10 ring atoms may contain a keto or —SO₂— group,
wherein such monocyclic or bicyclic heterocyclic moiety having 4 to 10 ring atoms may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
R₆ is selected from the group consisting of: hydrogen and C₁₋₃ unbranched alkyl;
R₇ is C₁₋₃ unbranched alkyl;
R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
wherein such saturated or unsaturated heterocyclic moiety having 4 to 8 ring atoms may contain an additional heteroatom selected from the group consisting of: N, O and S or may contain a group selected from the group consisting of: a keto or —SO₂— group,
wherein such saturated or unsaturated heterocyclic moiety having 4 to 8 ring atoms may be substituted with C₁₋₄ alkyl;
R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁-together with the nitrogen atom to which they are bonded—form a monocyclic, bicyclic or tricyclic alkyl or alkenyl group,
wherein such branched or unbranched C₁₋₈ alkyl and/or branched or unbranched C₁₋₈ alkenyl groups may contain one or more heteroatoms selected from the group consisting of: O, N, and S,
wherein such branched or unbranched C₁₋₈ alkyl and/or branched or unbranched C₁₋₈ alkenyl groups may contain a group selected from the group consisting of: keto and —SO₂-group and wherein such keto and —SO₂-group may be substituted with a hydroxy or amino group,
wherein such C₃₋₈cycloalkyl and/or C₃₋₈cycloalkenyl group may contain one or more ring heteroatoms selected from the group consisting of: O, N and S,
wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
wherein such monocyclic, bicyclic or tricyclic alkyl or alkenyl group may contain ring heteroatoms selected from the group consisting of: O, N and S,
wherein such monocyclic, bicyclic or tricyclic alkyl or alkenyl group may contain a group selected from the group consisting of: keto and SO₂,
wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 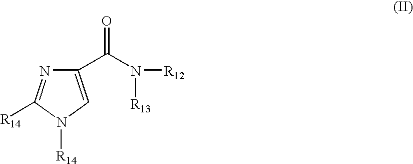
wherein
R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 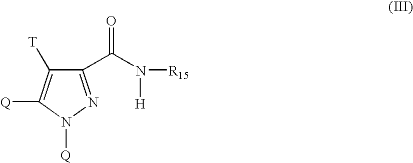
wherein
Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
T is selected from the group consisting of: hydrogen, C₁₋₃ alkyl and C₃₋₆ cycloalkyl which may contain from 1 to 3 heteroatoms selected from the group consisting of: N, O and S;
R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and a prodrug thereof, a tautomer thereof or a pharmaceutically acceptable salt thereof; and
e.) mixtures of any of the foregoing.

20. A method of treating a medical condition comprising the step of inhibiting a potassium K_{v1.3} channel by administering one or more compounds having a potassium K_{v1.3} channel inhibition of greater than about 40% and selected from the group consisting of:
a.) 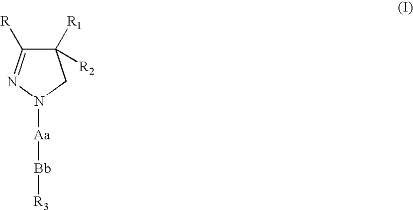
wherein:
R and R₁ are independently selected from the group consisting of: napthyl, phenyl, thienyl and pyridyl wherein phenyl, thienyl and pyridyl may be substituted with 1, 2 or 3 substitutents Y;
R₂ is selected from the group consisting of: hydrogen, hydroxy, C₁₋₃-alkoxy, acetyloxy and propionyloxy;
R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
Aa is selected from the group consisting of: substitutents of formulae (i), (ii), (iii), (iv), (v) and (vi) 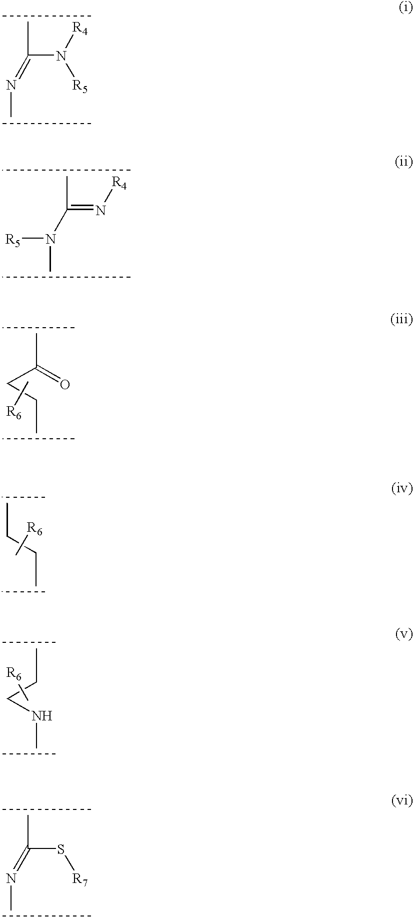
Bb is selected elected from the group consisting of: sulfonyl and carbonyl;
each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
R₄ is selected from the group consisting of: hydrogen, C₁₋₈ branched or unbranched alkyl and C₃₋₈ cycloalkyl; or R₄ is selected from the group consisting of: acetamido, dimethylamino, 2,2,2-trifluoroethyl, phenyl and pyridyl with the proviso that R₅ is hydrogen,
wherein such C₁₋₈ branched or unbranched alkyl and/or C₃₋₈ cycloalkyl alkyl group may be substituted with a hydroxyl group;
R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
wherein such C₁₋₈ branched or unbranched alkyl and/or C₃₋₈ cycloalkyl group may be substituted with hydroxyl and/or fluoro,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₃ alkyl, hydroxy, amino, monoalkylamino, dialkylamino or fluoro,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
wherein such phenyl, benzyl, pyridyl, thienyl, pyridylmethyl or phenethyl group may be substituted with 1, 2 or 3 of the substitutents Y,
wherein such monocyclic or bicyclic heterocyclic moiety having 4 to 10 ring atoms may contain one or more heteroatoms selected from the group consisting of: O, N and S,
wherein such monocyclic or bicyclic heterocyclic moiety having 4 to 10 ring atoms may contain a keto or —SO₂— group,
wherein such monocyclic or bicyclic heterocyclic moiety having 4 to 10 ring atoms may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
R₆ is selected from the group consisting of: hydrogen and C₁₋₃ unbranched alkyl;
R₇ is C₁₋₃ unbranched alkyl;
R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
wherein such saturated or unsaturated heterocyclic moiety having 4 to 8 ring atoms may contain an additional heteroatom selected from the group consisting of: N, O and S or may contain a group selected from the group consisting of: a keto or —SO₂— group,
wherein such saturated or unsaturated heterocyclic moiety having 4 to 8 ring atoms may be substituted with C₁₋₄ alkyl;
R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁-together with the nitrogen atom to which they are bonded—form a monocyclic, bicyclic or tricyclic alkyl or alkenyl group,
wherein such branched or unbranched C₁₋₈ alkyl and/or branched or unbranched C₁₋₈ alkenyl groups may contain one or more heteroatoms selected from the group consisting of: O, N, and S,
wherein such branched or unbranched C₁₋₈ alkyl and/or branched or unbranched C₁₋₈ alkenyl groups may contain a group selected from the group consisting of: keto and —SO₂-group and wherein such keto and —SO₂-group may be substituted with a hydroxy or amino group,
wherein such C₃₋₈ cycloalkyl and/or C₃₋₈ cycloalkenyl group may contain one or more ring heteroatoms selected from the group consisting of: O, N and S,
wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
wherein such monocyclic, bicyclic or tricyclic alkyl or alkenyl group may contain ring heteroatoms selected from the group consisting of: O, N and S,
wherein such monocyclic, bicyclic or tricyclic alkyl or alkenyl group may contain a group selected from the group consisting of: keto and SO₂,
wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 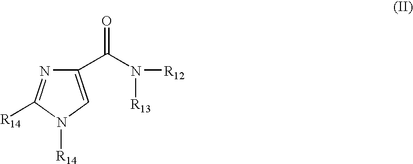
wherein
R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 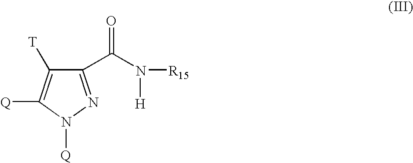
wherein
Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
T is selected from the group consisting of: hydrogen, C₁₋₃ alkyl and C₃₋₆ cycloalkyl which may contain from 1 to 3 heteroatoms selected from the group consisting of: N, O and S;
R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and a prodrug thereof, a tautomer thereof or a pharmaceutically acceptable salt thereof; and
e.) mixtures of any of the foregoing.

21. The method according to claim 20 wherein R₂ is hydrogen and wherein the 4-position of the 4,5 dihydro pyrazole ring is in the S-configuration.

22. A method of treating a medical condition comprising the step of inhibiting a potassium K_{v1.3} channel by administering one or more compounds selected from the group consisting of: 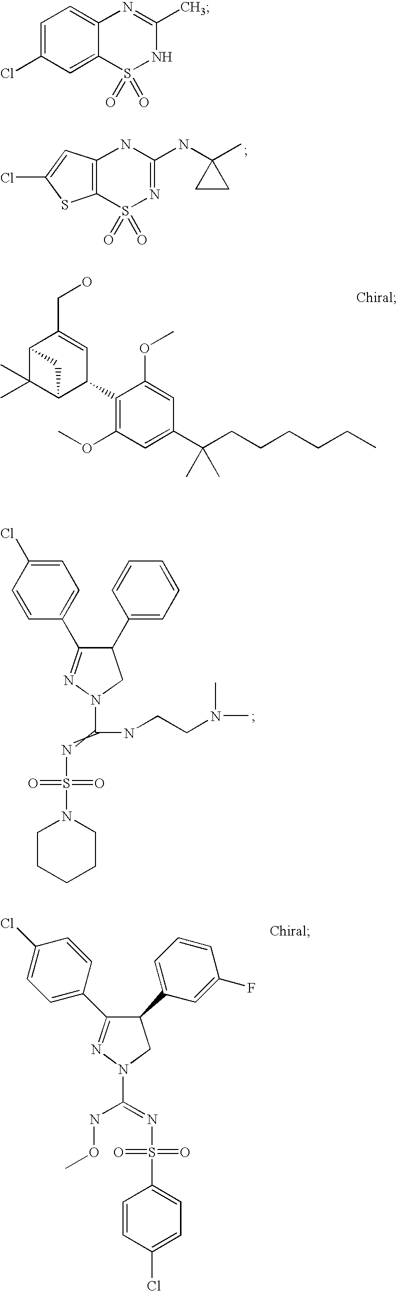 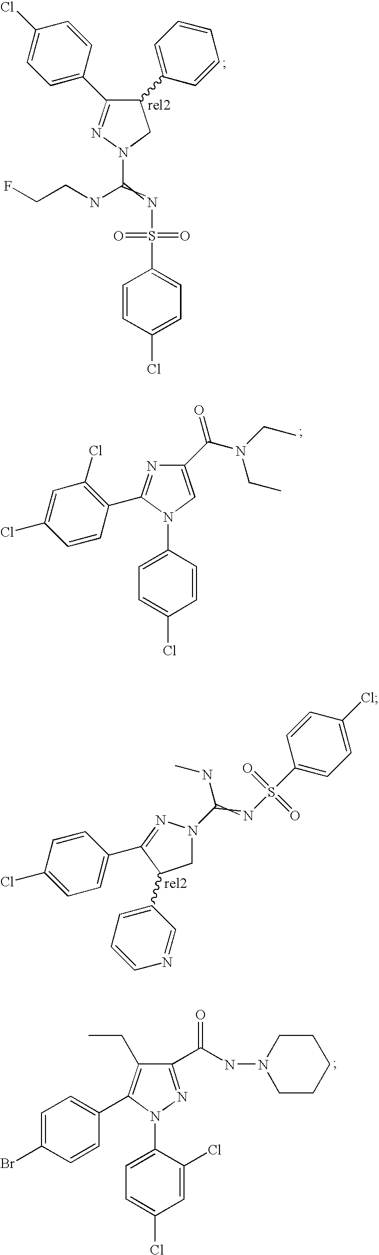 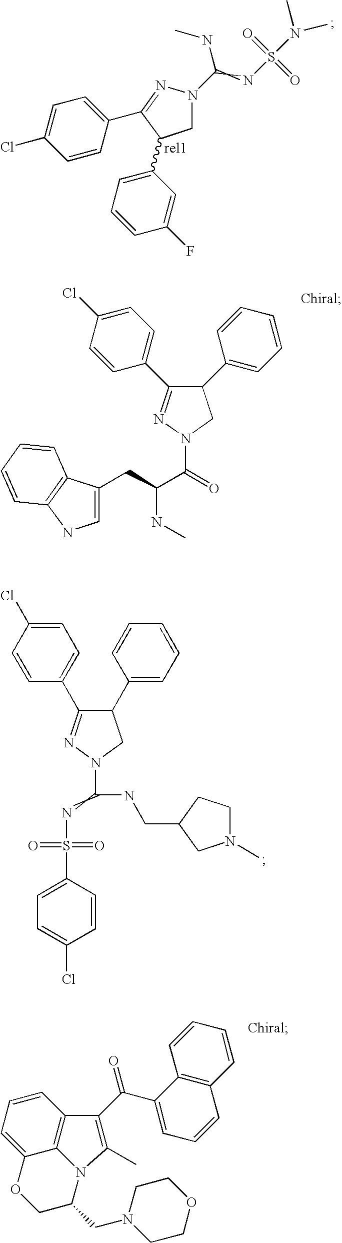 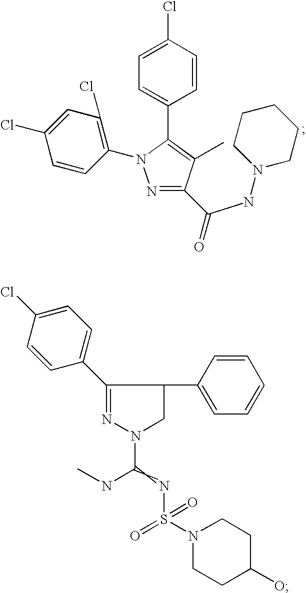
and mixtures thereof.

23. A pharmaceutical composition comprising a pharmacologically effective amount of one or more potassium K_{v1.3} channel inhibitor selected from the group consisting of:
a.) 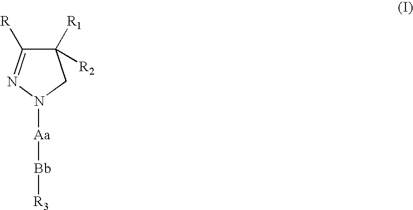
wherein:
R and R₁ are independently selected from the group consisting of: napthyl, phenyl, thienyl and pyridyl wherein phenyl, thienyl and pyridyl may be substituted with 1, 2 or 3 substitutents Y;
R₂ is selected from the group consisting of: hydrogen, hydroxy, C₁₋₃-alkoxy, acetyloxy and propionyloxy;
R₃ is selected from the group consisting of: C₁₋₈ branched or unbranched alkyl, C₃₋₁₀ cycloalkyl, C₃₋₈ alkenyl, C₅₋₁₀ bicycloalkyl, C₆₋₁₀ tricycloalkyl, C₅₋₈ cycloalkenyl, NR₁₀R₁₁, naphtyl, benzyl, phenyl, thienyl and pyridyl wherein benzyl, phenyl, thienyl and pyridiyl may be substituted with 1, 2 or 3 substitutents Y;
Aa is selected from the group consisting of: substitutents of formulae (i), (ii), (iii), (iv), (v) and (vi) 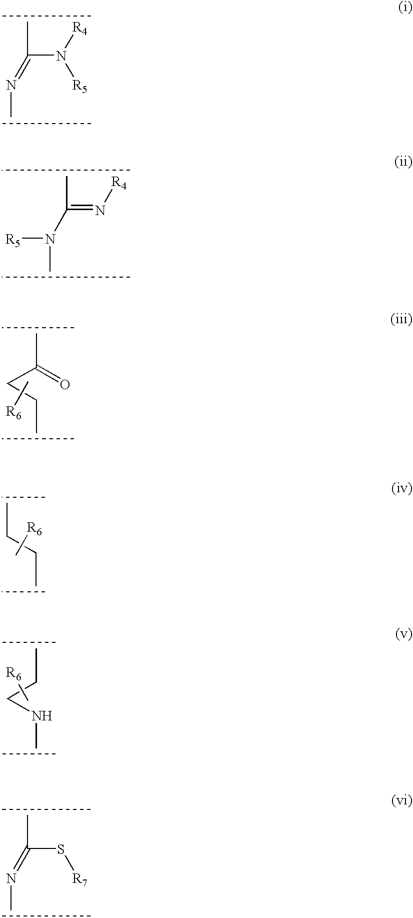
Bb is selected elected from the group consisting of: sulfonyl and carbonyl;
each Y is independently selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
R₄ is selected from the group consisting of: hydrogen, C₁₋₈ branched or unbranched alkyl and C₃₋₈ cycloalkyl; or R₄ is selected from the group consisting of: acetamido, dimethylamino, 2,2,2-trifluoroethyl, phenyl and pyridyl with the proviso that R₅ is hydrogen,
wherein such C₁₋₈ branched or unbranched alkyl and/or C₃₋₈ cycloalkyl alkyl group may be substituted with a hydroxyl group;
R₅ is selected from the group consisting of: hydrogen, C₁₋₈ branched or unbranched alkyl, C₃₋₈ cycloalkyl, C₂₋₁₀ branched or unbranched heteroalkyl, C₃₋₈ non-aromatic heterocycloalkyl, C₄₋₁₀ non-aromatic heterocycloalkyl-alkyl, amino, hydroxy, phenoxy, benzyloxy, C₁₋₈ alkoxy, C₃₋₈ alkenyl, C₅₋₈ cycloalkenyl, C₆₋₉ cycloalkenylalkyl, imidazolylalkyl, phenyl, benzyl, pyridyl, thienyl, pyridylmethyl and phenethyl; or R₅ is NR₈R₉ with the proviso that R₄ is H or methyl; or R₄ and R₅ together with the nitrogen atom to which they are bonded form a saturated or unsaturated, monocyclic or bicyclic heterocyclic moiety having 4 to 10 ring atoms,
wherein such C₁₋₈ branched or unbranched alkyl and/or C₃₋₈ cycloalkyl group may be substituted with hydroxyl and/or fluoro,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain one or more heteroatoms selected from the group consisting of: O, N and S,
wherein such C₂₋₁₀ branched or unbranched heteroalkyl, C₃₋₈ non-aromatic heterocycloalkyl and/or C₄₋₁₀ non-aromatic heterocycloalkyl-alkyl groups may contain a SO₂— group,
wherein such C₂₋₁₀ branched or unbranched heteroalkyl group, C₃₋₈ non-aromatic heterocycloalkyl group and/or C₄₋₁₀ non-aromatic heterocycloalkylalkyl group may be substituted with keto, trifluoromethyl, C₁₋₃ alkyl, hydroxy, amino, monoalkylamino, dialkylamino or fluoro,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain one or more heteroatoms selected from the group consisting of: O, N and S,
wherein such amino, hydroxy, phenoxy, benzyloxy, C₁₋₈ alkoxy, C₃₋₈ alkenyl, C₅₋₈ cycloalkenyl, C₆₋₉ cycloalkenylalkyl may contain a keto or —SO₂— group,
wherein such C₁₋₈ alkoxy, C₃₋₈ alkenyl and C₅₋₈ cycloalkenyl groups may be substituted with a hydroxy group, a trifluoromethyl group, an amino group, a monoalkylamino group or dialkylamino group or a fluoro atom,
wherein such phenyl, benzyl, pyridyl, thienyl, pyridylmethyl or phenethyl group may be substituted with 1, 2 or 3 of the substitutents Y,
wherein such monocyclic or bicyclic heterocyclic moiety having 4 to 10 ring atoms may contain one or more heteroatoms selected from the group consisting of: O, N and S,
wherein such monocyclic or bicyclic heterocyclic moiety having 4 to 10 ring atoms may contain a keto or —SO₂— group,
wherein such monocyclic or bicyclic heterocyclic moiety having 4 to 10 ring atoms may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;
R₆ is selected from the group consisting of: hydrogen and C₁₋₃ unbranched alkyl;
R₇ is C₁₋₃ unbranched alkyl;
R₈ and R₉ are the same or different and are selected elected from the group consisting of: C₂₋₄ alkyl and C₂₋₄ trifluoroalkyl; or R₈ is methyl with the proviso that R₉ is C₂₋₄ alkyl; or R₈ and R₉—together with the nitrogen atom to which they are bonded—form a saturated or unsaturated heterocyclic moiety having 4 to 8 ring atoms,
wherein such saturated or unsaturated heterocyclic moiety having 4 to 8 ring atoms may contain an additional heteroatom selected from the group consisting of: N, O and S or may contain a group selected from the group consisting of: a keto or —SO₂— group,
wherein such saturated or unsaturated heterocyclic moiety having 4 to 8 ring atoms may be substituted with C₁₋₄ alkyl;
R₁₀ and R₁₁ are independently selected from the group consisting of: hydrogen, branched or unbranched C₁₋₈ alkyl, branched or unbranched C₁₋₈ alkenyl, C₃₋₈ cycloalkyl, C₃₋₈ cycloalkenyl, naphtyl and phenyl; or R₁₀ and R₁₁—together with the nitrogen atom to which they are bonded—form a monocyclic, bicyclic or tricyclic alkyl or alkenyl group,
wherein such branched or unbranched C₁₋₈ alkyl and/or branched or unbranched C₁₋₈ alkenyl groups may contain one or more heteroatoms selected from the group consisting of: O, N, and S,
wherein such branched or unbranched C₁₋₈ alkyl and/or branched or unbranched C₁₋₈ alkenyl groups may contain a group selected from the group consisting of: keto and —SO₂-group and wherein such keto and —SO₂-group may be substituted with a hydroxy or amino group,
wherein such C₃₋₈ cycloalkyl and/or C₃₋₈ cycloalkenyl group may contain one or more ring heteroatoms selected from the group consisting of: O, N and S,
wherein such C₃₋₈ cycloalkyl and/or C₃₋₈ cycloalkenyl group may be substituted with hydroxy, C₁₋₃ alkyl, —SO₂—, keto, amino, C₁₋₃ monoalkylamino and/or C₁₋₃ dialkylamino,
wherein such phenyl group may be substituted with 1, 2 or 3 substitutents Y with the proviso that R₁₁ is selected from the group consisting of: hydrogen, branched or unbranched C₁₋₅ alkyl group wherein such branched or unbranched C₁₋₅ alkyl group may contain one or more heteroatoms selected from the group consisting of: O, N and S or wherein such branched or unbranched C₁₋₅ alkyl group may contain SO₂— and wherein such branched or unbranched C₁₋₅ alkyl group may be substituted with a hydroxy, keto or amino group,
wherein such monocyclic, bicyclic or tricyclic alkyl or alkenyl group may contain ring heteroatoms selected from the group consisting of: O, N and S,
wherein such monocyclic, bicyclic or tricyclic alkyl or alkenyl group may contain a group selected from the group consisting of: keto and SO₂,
wherein such monocyclic, bicyclic or tricyclic alkyl or alkenyl group may be substituted with hydroxy, C₁₋₃ alkyl, SO₂—, keto, amino, C₁₋₃ monoalkylamino, C₁₋₃ dialkylamino, pyrrolidinyl, or piperidinyl,
wherein such monocyclic, bicyclic or tricyclic alkyl or alkenyl group may contain an annelated phenyl group which annelated phenyl group may be substituted with 1 or 2 substitutents Y; and
a prodrug thereof, a tautomer thereof or a pharmaceutically acceptable salt thereof;
b.) 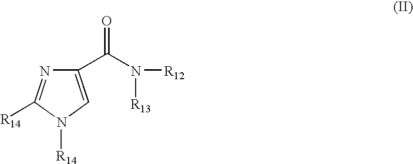
wherein
R₁₂ and R₁₃ are independently selected from the group consisting of: hydrogen, C₁₋₃ alkyl and C₃₋₆ cycloalkyl which may contain from 1 to 3 heteroatoms selected from the group consisting of: N, O and S;
R₁₄ is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl; and
a prodrug thereof, a tautomer thereof or a pharmaceutically acceptable salt thereof;
c.) 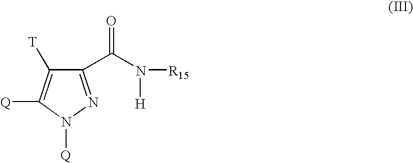
wherein
Q is phenyl which may be substituted with 1, 2 or 3 substitutents Z which can be the same or different and wherein Z is selected from the group consisting of: C₁₋₃-alkyl, C₁₋₃-alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl;
T is selected from the group consisting of: hydrogen, C₁₋₃ alkyl and C₃₋₆ cycloalkyl which may contain from 1 to 3 heteroatoms selected from the group consisting of: N, O and S;
R₁₅ is selected from the group consisting of: C₁₋₃ alkyl and C₃₋₆ cycloalkyl which may contain from 1 to 3 heteroatoms selected from the group consisting of: N, O and S; and
a prodrug thereof, a tautomer thereof or a pharmaceutically acceptable salt thereof;
d.) Diazoxide, NN414, R(+)-WIN55212-2, HU-308, Rimonabant, SR-147778; and
a prodrug thereof, a tautomer thereof or a pharmaceutically acceptable salt thereof; and
e.) mixtures of any of the foregoing.

24. The method according to claim 23 wherein R₂ is hydrogen and wherein the 4-position of the 4,5 dihydro pyrazole ring is in the S-configuration.

25. A pharmaceutical composition comprising a pharmacologically effective amount of one or more potassium K_{v1.3} channel inhibitor selected from the group consisting of: 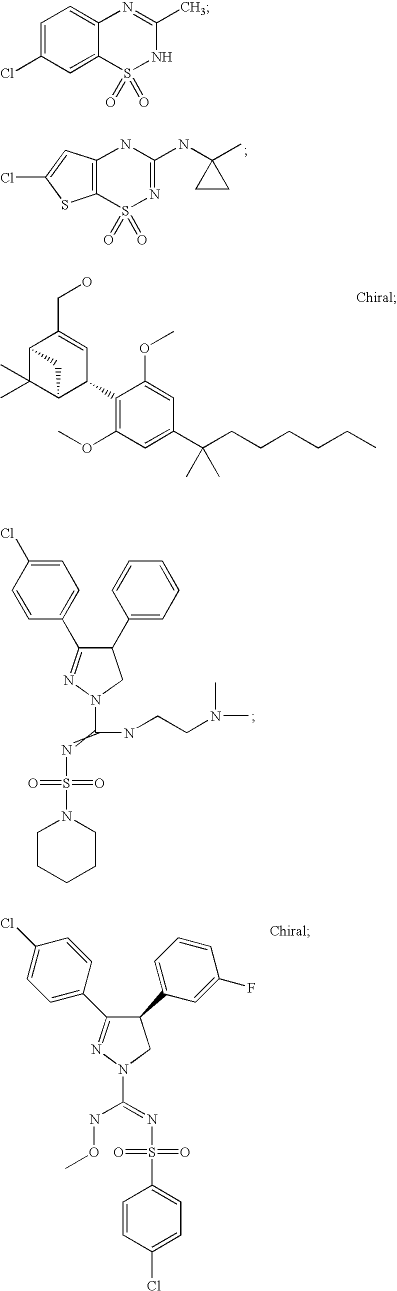 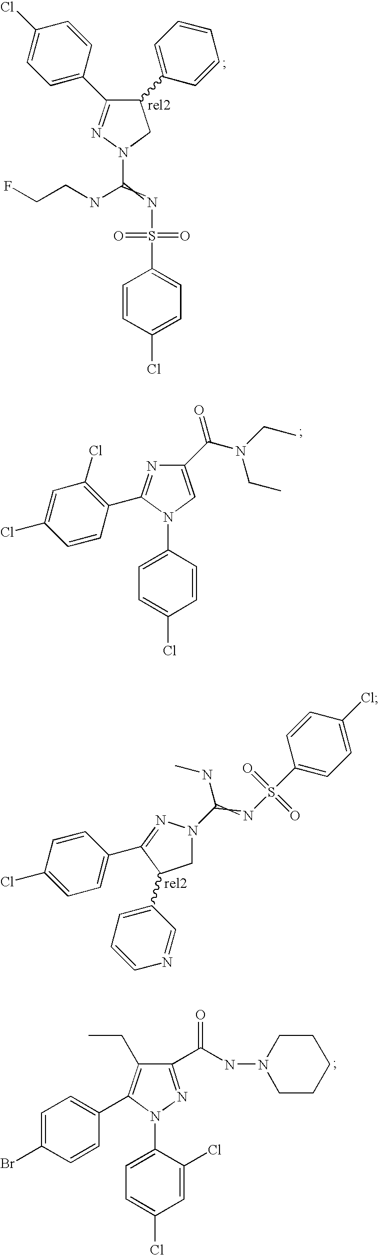 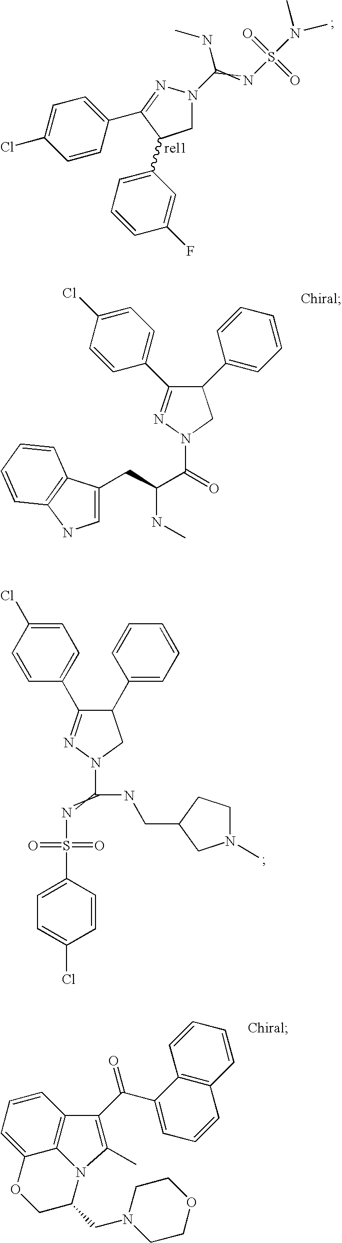 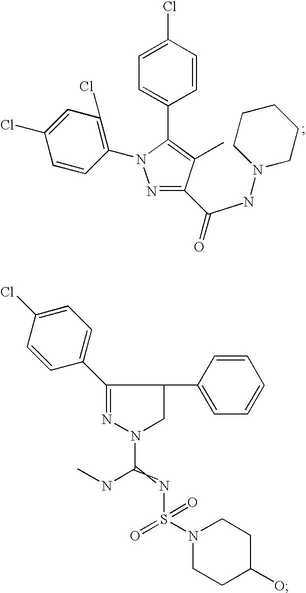
and mixtures thereof.

26. A pharmaceutical composition comprising one or more potassium K_{v1.3} channel inhibiting compounds wherein at least one of the one or more compounds inhibits the potassium K_{v1.3} channel by greater than about 40%.

27. The pharmaceutical composition according to claim 26 wherein the compounds are selected from the group consisting of: 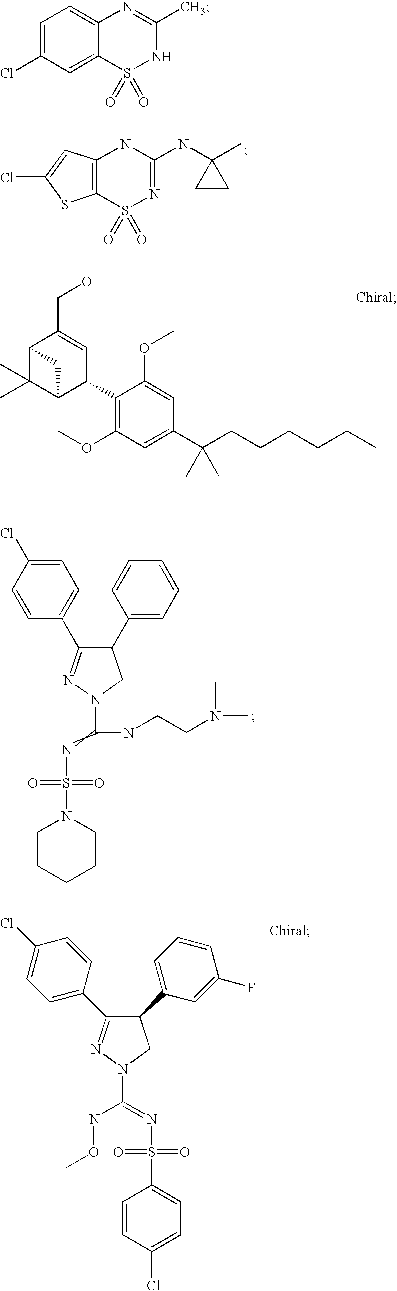 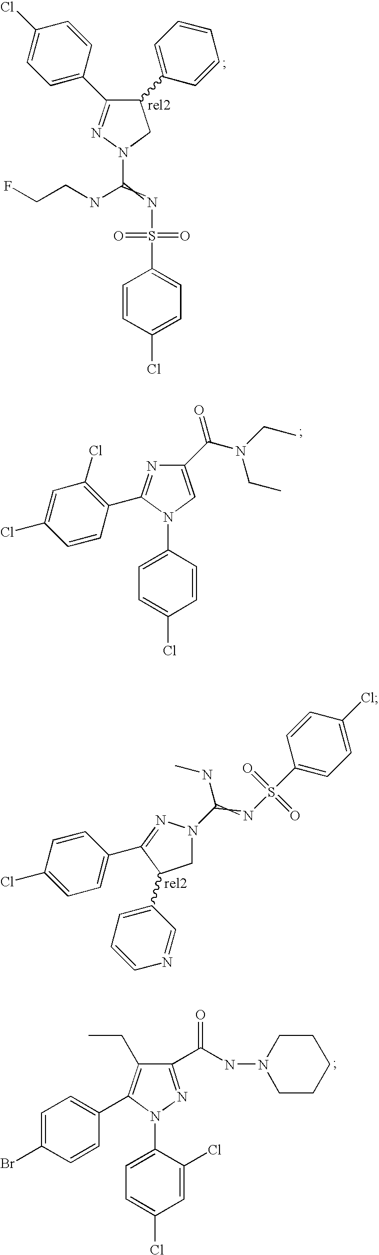 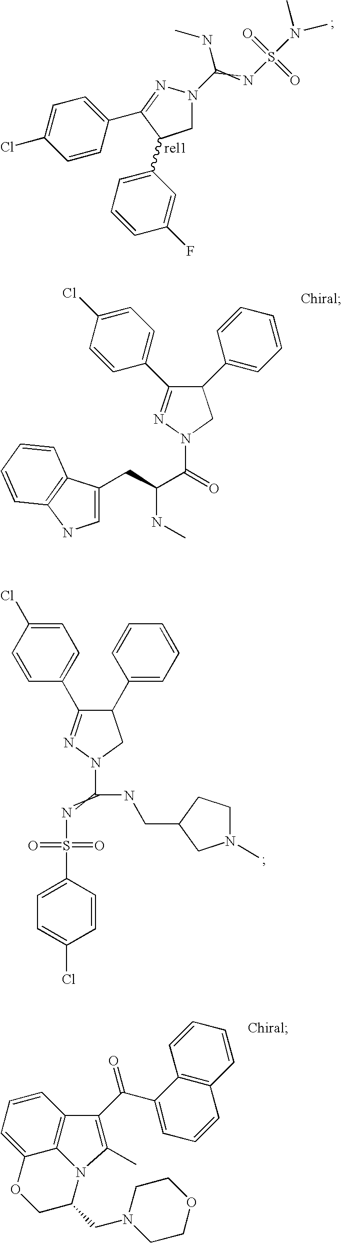 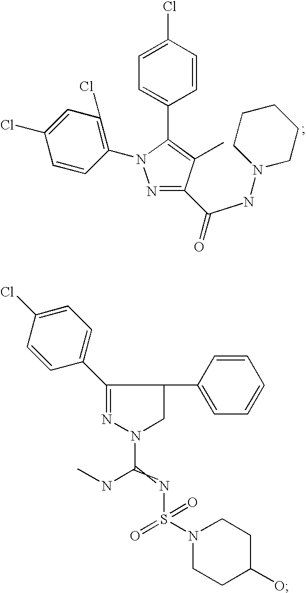
and mixtures thereof.

28. A pharmaceutical composition comprising one or more potassium K_{v1.3} channel inhibiting compounds wherein at least one of the one or more compounds inhibits the potassium K_{v1.3} channel by greater than about 60%.

29. The pharmaceutical composition according to claim 28 wherein the compounds are selected from the group consisting of: 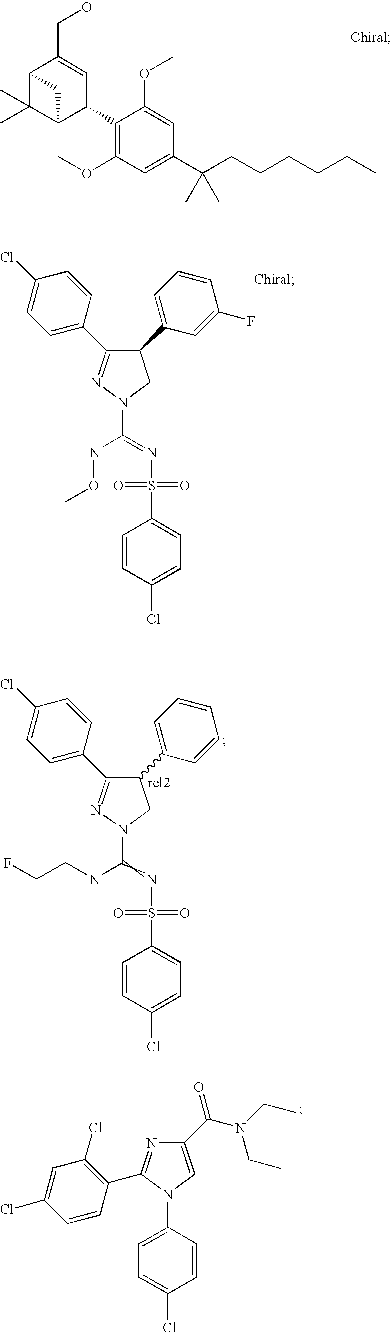 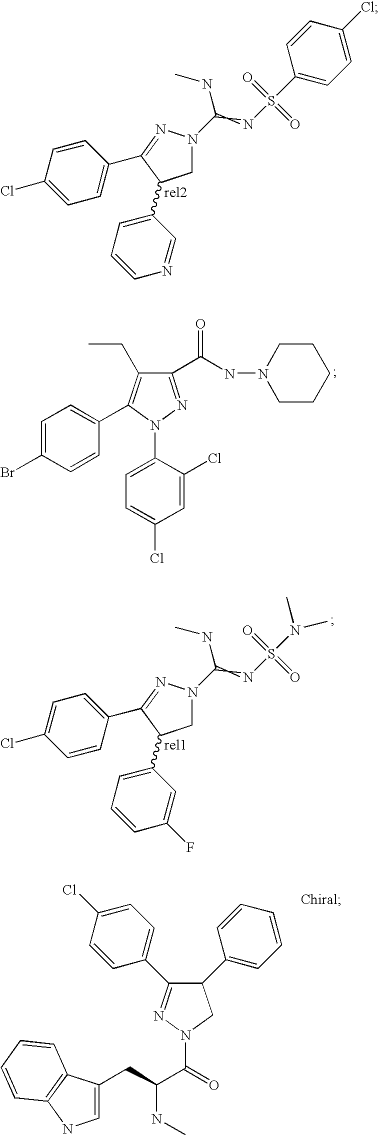 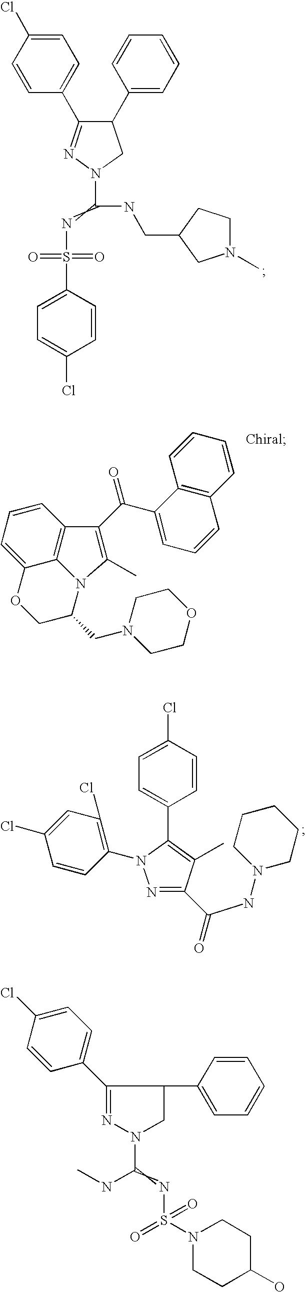
and mixtures thereof.

30. A pharmaceutical composition comprising one or more potassium K_{v1.3} channel inhibiting compounds wherein at least one of the one or more compounds inhibits the potassium K_{v1.3} channel by greater than about 80%.

31. The pharmaceutical composition according to claim 30 wherein the compounds are selected from the group consisting of: 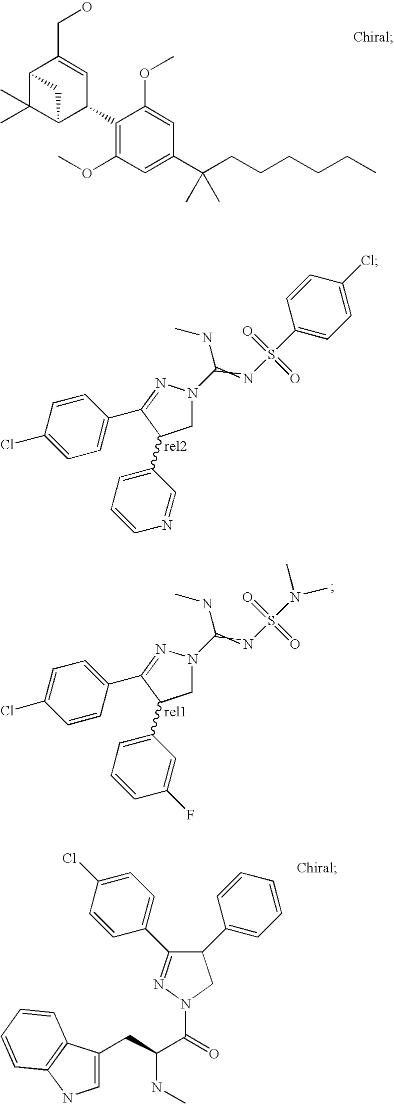 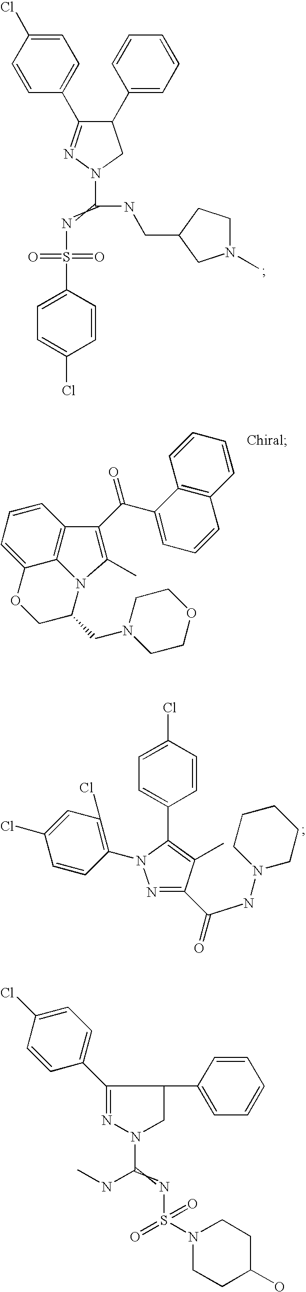
and mixtures thereof.

32. A pharmaceutical composition comprising one or more potassium K_{v1.3} channel inhibiting compounds wherein at least one of the one or more compounds inhibits the potassium K_{v1.3} channel by greater than about 90%.

33. The pharmaceutical composition according to claim 32 wherein the compounds are selected from the group consisting of: 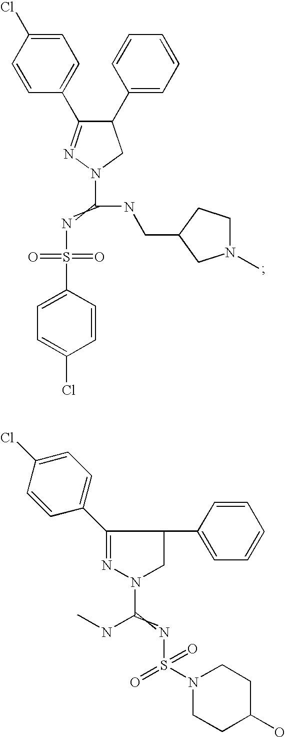
and mixtures thereof.

34. A pharmaceutical composition comprising one or more potassium K_{v1.3} channel inhibiting compounds wherein at least one of the one or more compounds inhibits the potassium K_{v1.3} channel by greater than about 95%.

35. The pharmaceutical composition according to claim 34 wherein the compounds are selected from the group consisting of: [chemical structure]
